SEQUENCE LISTING (14 sequences)

SEQ ID NO: 1 - protein, 5 aa, synthetic construct
NYWMH

SEQ ID NO: 2 - protein, 17 aa, synthetic construct
ATYRGHSDTYYNQKFKG

SEQ ID NO: 3 - protein, 12 aa, synthetic construct
GAIYDGYDVLDN

SEQ ID NO: 4 - protein, 11 aa, synthetic construct
SASQDISNYLN

SEQ ID NO: 5 - protein, 7 aa, synthetic construct
YTSNLHS

SEQ ID NO: 6 - protein, 9 aa, synthetic construct
QQYRKLPWT

SEQ ID NO: 7 - protein, 121 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 8 - protein, 108 aa, synthetic construct
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 9 - protein, 451 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 451 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 451 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESDGQPENNYKTTPPVLDSDGSFFLYSKLTVDKSRWQQGNVFSCSVMHEALHNHYTQKSLSLSPGK

SEQ ID NO: 13 - protein, 451 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPEDNYKTTPPVLDSDGSFFLYSKLTVDKSRWQQGNVFSCSVMHEALHNHYTQKSLSLSPGK

SEQ ID NO: 14 - protein, 451 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESDGQPEDNYKTTPPVLDSDGSFFLYSKLTVDKSRWQQGNVFSCSVMHEALHNHYTQKSLSLSPGK

(19) United States
(12) Patent Application Publication
(10) Pub. No.: US 2024/0369568 A1
(43) Pub. Date: Nov. 7, 2024

(54) Biopharmaceutical Compositions and Stable Isotope Labeling Peptide Mapping Method

(71) Applicant: GlaxoSmithKline Intellectual Property Development Limited, Stevenage (GB)

(72) Inventors: Tyler Keith DAVIS, Collegeville, PA (US); Hillary Amber SCHUESSLER, Collegeville, PA (US)

(73) Assignee: GlaxoSmithKline Intellectual Property Development Limited, Stevenage (GB)

(21) Appl. No.: 18/291,272

(22) PCT Filed: Aug. 2, 2022
(86) PCT No.: PCT/IB2022/057173

§ 371 Date: Jan. 23, 2024

Related U.S. Application Data
(60) Provisional application No. 63/228,951, filed on Aug. 3, 2021.

(51) Int. Cl. G01N 33/68

(52) U.S. Cl. CPC G01N 33/6854; G01N 33/6848; G01N 2333/976; G01N 2440/00

(57) ABSTRACT

Disclosed herein are stable isotope labeling (SIL) peptide mapping methods for accurate and sensitive conjugation site quantitation. Also disclosed herein are compositions comprising antibody drug conjugates (ADCs) that target BCMA.

### CROSS-REFERENCE TO RELATED APPLICATION

The subject application claims priority to U.S. Patent Application No. 63/228,951, filed on Aug. 3, 2021, the entirety of which is incorporated herein by reference.

### SEQUENCE LISTING

The instant application contains a Sequence Listing which has been submitted electronically in XML format and is hereby incorporated by reference in its entirety. Said XML file is named 054624_09_5031_Sequence_Listing.xml, was created on Jul. 25, 2022, and is 15,855 bytes in size.

### FIELD

Disclosed herein are methods that determine site-specific conjugation levels of cysteine-conjugated antibody drug conjugates with a small-molecule cytotoxic payload, e.g., MMAF or MMAE, using stable isotope labeling (SIL) peptide mapping.

### BACKGROUND

Antibody drug conjugates (ADCs) are a growing class of biopharmaceuticals that combine the specificity of monoclonal antibodies (mAbs) with the potency of cytotoxic small-molecule drugs for targeted oncology therapies (Hafeez et al., Antibody-Drug Conjugates for Cancer Therapy. Molecules. 2020, 25, 4764; and Boni et al., The Resurgence of Antibody Drug Conjugates in Cancer Therapeutics: Novel Targets and Payloads. Am Soc Clin Oncol Educ Book. 2020, 40, 1-17). Small-molecule payloads are typically conjugated through cysteine or lysine protein residues resulting in heterogeneous mixtures of different drug-loaded (DL) species (Ponziani et al., Antibody-Drug Conjugates: The New Frontier of Chemotherapy. Int. J. Mol. Sci. 2020, 21, 5510). The overall drug-to-antibody ratio (DAR) of ADCs has been identified as a critical quality attribute (CQA) due to its effect on drug potency and efficacy (Li et al., Impact of Physiologically Based Pharmacokinetics, Population Pharmacokinetics and Pharmacokinetics/Pharmacodynamics in the Development of Antibody-Drug Conjugates. The Journal of Clinical Pharmacology. 2020, 60, 105-119). As a result, ADCs require the analytical challenge of characterizing these drug-loaded species in addition to protein sequence and post-translational modifications (PTMs) typically characterized for mAb biopharmaceuticals.

Several analytical methods can determine a global DAR for ADCs including hydrophobic interaction chromatography (HIC) (Bobaly et al., Optimization of non-linear gradient in hydrophobic interaction chromatography for the analytical characterization of antibody-drug conjugates. Journal of Chromatography A. 2017, 1481, 82-91), hydrophilic interaction chromatography (HILIC) (D'Atri et al., Characterization of an antibody-drug conjugate by hydrophilic interaction chromatography coupled to mass spectrometry. Journal of Chromatography B. 2018, 1080, 37-41), capillary gel electrophoresis (CGE) (Lechner et al., Insights from capillary electrophoresis approaches for characterization of monoclonal antibodies and antibody drug conjugates in the period 2016-2018. Journal of Chromatography B. 2019, 1122-1123, 1-170), and native and sub-unit liquid chromatography mass spectrometry (LC-MS) (Zhu et al., Current LC-MS-based strategies for characterization and quantification of antibody-drug conjugates. Journal of Pharmaceutical Analysis. 2020, 10, 209-220). These methods can also provide qualitative information about conjugation site locations; however, site-specific conjugation levels have been difficult to assess.

Liquid chromatography tandem mass spectrometry (LC-MS/MS) analysis of enzymatic digests (peptide mapping) is a widely used analytical method to characterize protein sequence and quantify post-translational modifications (PTMs) of biopharmaceuticals by performing relative quantitation between native and modified versions of peptides. MMAF-conjugated peptides complicate this approach due to the relatively large mass and retention time differences between native and conjugated peptides due to the addition of the hydrophobic drug payload. These differences result in peptide pairs with vastly different ionization efficiencies which makes relative quantitation between them unsuitable.

As a result, most ADC peptide mapping applications have been qualitative in nature by only confirming conjugation site locations, but few attempts to quantify the level of conjugation at these sites have been described. One method described by Q. Luo et. al. (Structural Characterization of a Monoclonal Antibody-Maytansinoid Immunoconjugate. Analytical Chemistry. 2016, 88, 695-702) involved analyzing an unconjugated mAb intermediate sample along with ADC samples. The unconjugated peak areas detected in the mAb sample were compared to those in the ADC samples and any loss of area was attributed to conjugation at that peptide site. However, this method does not normalize for sample preparation variations between the mAb and ADC samples and cannot account for multiple conjugation sites on a single peptide, such as the heavy chain hinge peptide for cysteine-conjugated ADCs.

Another method described by L. Chen, et. al. (In-depth structural characterization of Kadcyla® (ado-trastuzumab emtansine) and its biosimilar candidate. mAbs. 2016, 8, 1210-1223) attempted to correct for sample preparation variability by normalizing conjugated peptide peak areas to the peak area of a known addition of the peptide leucine enkephalin. However, this method only provides relative conjugation quantitation between samples and not a true site-occupancy percentage for a given conjugation site.

A third method described by H. Sang, et. al. (Conjugation site analysis of antibody-drug-conjugates (ADCs) by signature ion fingerprinting and normalized area quantitation approach using nano-liquid chromatography coupled to high resolution mass spectrometry. Analytica Chimica Acta. 2017, 955, 67-78) attempted to account for ionization differences by normalizing the peak area of a conjugated peptide with the peak area of its respective unconjugated peptide. This ratio was then multiplied by a relative ionization intensity factor calculated by dividing the slopes of unconjugated and conjugated peptide calibration curves to yield a normalized ratio. Site conjugation levels were then calculated as a function of this normalized ratio. However, this method requires the analysis of standards to construct calibration curves for all conjugation site peptide pairs. Furthermore, the conjugation level equation as described does not account for multiple conjugation sites on a single peptide.

Stable isotope labeling (SIL) is the process of incorporating heavy isotope atoms into analytes of interest which results in mass changes that can then be detected by mass spectrometry. SIL peptide mapping is a popular method in the field of proteomics to provide relative quantitation of proteins in differentially labeled samples (Liu et al., Advances and applications of stable isotope labeling-based methods for proteome relative quantitation. Trends in Anal. Chem. 2020, 124, 115815) but has also been applied to protein PTM characterization. Liu et al. (Accurate Determination of Protein Methionine Oxidation by Stable Isotope Labeling and LC-MS Analysis. Anal. Chem. 2013, 85, 11705-11709) investigated methionine oxidation levels by reacting a mAb sample with oxygen-18 hydrogen peroxide to completely oxidize methionines of interest. Relative quantitation was then performed between the natural (+16 Da) and SIL (+18 Da) versions of oxidized peptides using isotope peak areas.

Therefore, there is a need in the art to provide improved analysis methods for ADCs.

### SUMMARY

According to a first aspect of the disclosure, there is provided a method (e.g., an analytical method) comprising:
(i) conjugation of unoccupied cysteine sites of a cysteine-conjugated antibody drug conjugate (ADC) using an isotopically-labeled cytotoxin containing a carbonyl group and a reductant to produce an isotopically-labeled ADC sample; and
(ii) peptide mapping the sample.

According to a further aspect of the disclosure, there is provided a composition comprising an anti-BCMA antibody conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the percentage drug load at LC C214 is between about 56% to about 80%, the percentage drug load at HC C224 is between about 58% to about 81%, the percentage drug load of HC hinge DL2 at HC C230 and C233 is between about 15% to about 46%, and/or the percentage drug load of HC hinge DL1 at HC C230 or HC C233 is between about 11% to about 15%.

According to a further aspect of the disclosure, there is provided a pharmaceutical composition comprising the composition as described herein and at least one pharmaceutically acceptable excipient.

According to a further aspect of the disclosure, there is provided a formulation comprising the pharmaceutical composition as described herein comprising the ADC at about 20 mg/mL to about 60 mg/mL, citrate buffer at about 10 mM to about 30 mM, trehalose at about 120 mM to about 240 mM, EDTA at about 0.01 mM to about 0.1 mM, polysorbate 20 or polysorbate 80 at about 0.01% to about 0.05%, at a pH of about 5.9 to about 6.5.

According to a further aspect of the disclosure, there is provided a method of treating cancer comprising administering to a subject in need thereof a therapeutically effective amount of a composition or formulation as disclosed herein.

According to a further aspect of the disclosure, there is provided a composition or formulation as disclosed herein for use in the treatment of cancer.

According to a further aspect of the disclosure, there is provided the use of a composition as disclosed herein in the manufacture of a medicament for use in the treatment of cancer.

According to a further aspect of the disclosure, there is provides a method of determining conjugation levels of cysteine-conjugated antibody drug conjugates comprising: reducing the antibody drug conjugates to form reduced antibody drug conjugates; conjugating the reduced antibody drug conjugates with an isotypically-labeled cytotoxin to form isotopically-labeled antibody drug conjugates; producing isotopically-labeled conjugated peptides from the isotopically-labeled antibody drug conjugates and performing peptide mapping on the isotopically-labeled conjugated peptides; detecting mass-to-charge ratios for the isotopically-labeled conjugated peptides; and comparing the mass-to-charge ratios of the isotopically-labeled conjugated peptides to mass-to charge ratios for non-isotopically-labeled conjugated peptides to determine the conjugation levels of cysteine-conjugated antibody drug conjugates. In an embodiment, the cytotoxin is MMAF or MMAE. In another embodiment, the cysteine-conjugated antibody drug conjugates are first reduced by a reductant and then conjugated with the isotopically-labeled cytotoxin. In an embodiment, the reductant is dithiothreitol (DTT) or tris(2-carboxyethyl)phosphine (TCEP). In another embodiment, excess reductant is removed prior to the peptide mapping by eluting the sample through a size exclusion chromatography column. In yet another embodiment, conjugation occurs by reacting the cysteine-conjugated antibody drug conjugates with the isotopically-labeled cytotoxin. In another embodiment, excess isotopically-labeled cytotoxin is removed prior to the peptide mapping by eluting the sample through a size exclusion chromatography column. In yet another embodiment, the peptide mapping comprises using liquid chromatography tandem mass spectrometry (LC-MS/MS) analysis. In some embodiments, the peptide mapping comprises denaturing the sample, reducing remaining disulfide bonds, and alkylating resulting free sulfhydryls. In another embodiment, the peptide mapping comprises enzymatically digesting the sample to produce isotopically-labeled conjugated peptides and optionally quenching the enzymatic digestion by addition of a strong acid. In some embodiments, the method comprises reacting cytotoxin with isotopically-labeled water to produce the isotopically-labeled cytotoxin. In another embodiment, the cytotoxin is reacted with isotopically-labeled water in acetonitrile. In yet another embodiment, the cysteine-conjugated antibody drug conjugates are belantamab mafodotin.

### BRIEF DESCRIPTION OF THE DRAWINGS

FIG. 1 depicts the UV chromatogram and MS spectrum of stable isotope labeled MMAF.

FIG. 2 depicts the reduced LC-MS spectra for belantamab mafodotin light and heavy chains before and after stable isotope labeling.

FIG. 3 depicts a schematic representation of the heterogenous mixture of drug-loaded species in belantamab mafodotin.

FIG. 4 compares the XICs for the light chain, heavy chain fab, and heavy chain hinge peaks detected from standard and stable isotope labeled peptide mapping.

FIG. 5 depicts representative MS spectra for labeled and unlabeled conjugated light chain peptide. Natural isotope ratios used to calculate isotopomer contributions are labeled.

FIG. 6 depicts representative MS spectra for labeled and unlabeled conjugated heavy chain Fab peptide. Natural isotope ratios used to calculate isotopomer contributions are labeled.

FIG. 7 depicts representative MS spectra for labeled and unlabeled conjugated heavy chain hinge peptide. Natural isotope ratios used to calculate isotopomer contributions are labeled.

FIG. 8 compares the linearity response curves between standard and stable isotope labeled peptide mapping for all conjugation sites.

FIG. 9 compares Standard and SIL Peptide Mapping Calculated DARs and Theoretical DARs for Belantamab Mafodotin Linearity Samples

FIG. 10 depicts conjugation values for belantamab mafodotin differential DAR samples

FIG. 11 compares SIL Peptide Mapping Calculated DARs and Theoretical HIC DARs for Belantamab Mafodotin Differential DAR Samples FIG. 12 depicts the analytical and prep-scale hydrophobic interaction chromatography traces used to collect drug-load fractions.

FIG. 13 demonstrates representative NR-CGE Electropherograms of Purified DL0, DL2, DL4a, DL4b, DL6, and DL8

FIG. 14 shows intact mass spectra of purified DL0, DL2, DL4a, DL4b, DL6, and DL8 drug-load variants.

FIG. 15 shows reduced mass spectra of heavy (A) and light chain (B) of purified DL0, DL2, DL4a, DL4b, DL6, and DL8 drug-load variants.

FIG. 16 shows capillary differential scanning calorimetry (DSC) traces of purified DL0, DL2, DL4a, DL4b, DL6, and DL8 drug-load variants.

### DETAILED DESCRIPTION

### Stable Isotope Labelling (SIL) Peptide Mapping Method

The term “about” or “approximately” can mean within an acceptable error range for the particular value as determined by one of ordinary skill in the art, which will depend in part on how the value is measured or determined, e.g., the limitations of the measurement system.

For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

The present disclosure provides methods for determining site-specific conjugation levels of cysteine-conjugated antibody drug conjugates with a small-molecule payload, e.g. cytotoxin, using stable isotope labeling peptide mapping LC-MS/MS analysis.

According to a first aspect of the disclosure, there is provided an analytical method comprising:
(i) conjugation of unoccupied cysteine sites of a cysteine-conjugated antibody drug conjugate (ADC) using an isotopically-labeled cytotoxin containing a carbonyl group and a reductant to produce an isotopically-labeled ADC sample; and
(ii) peptide mapping the sample.

In one embodiment, the cytotoxin contains a carbonyl group, e.g. a carbonyl oxygen with a double bond.

In one embodiment, the cytotoxin is Monomethyl auristatin F (MMAF) or Monomethyl auristatin E (MMAE).

Liquid chromatography tandem mass spectrometry (LC-MS/MS) analysis of enzymatic digests (peptide mapping) is an analytical method widely known to those skilled in the art to characterize protein sequence and quantify PTMs of biopharmaceuticals by performing relative quantitation between native and modified versions of peptides. The analytical methods described herein reduce including, eliminate, the differential ionization efficiency problem associated with methods of the prior art by conjugating unoccupied cysteine sites with stable isotopically-labeled cytotoxin to produce peptide pairs with identical retention times and minimal differences in mass and hydrophobicity, thus allowing relative quantitation in parallel with other monitored post-translational modifications (PTMs) in one multi-attribute analytical method (MAM).

Stable isotope labeling (SIL) is the process of incorporating heavy-isotope atoms (e.g., carbon-13, nitrogen-15, oxygen-18) into analytes of interest, which results in mass changes that can be detected by mass spectrometry. The peptide mapping method described herein reduces, including eliminates, the peptide differential ionization efficiency problem by labeling available conjugation sites with isotopically labeled cytotoxin to produce conjugation-site peptide pairs with identical retention times and minimal mass differences. This method allows for accurate site-specific conjugation level quantitation and provides the first known example of “bottom-up” DAR characterization in parallel with protein sequence and PTM characterization in one multi-attribute analytical method (MAM).

In one embodiment, the analytical method comprises reacting cytotoxin with isotopically-labeled water (e.g., H₂¹⁸O) to produce the isotopically-labeled cytotoxin. In one embodiment, the isotopically labelled water undergoes a solvent exchange with the cytotoxin containing a carbonyl group (oxygen with double bond, e.g., a ketone) to create an isotopically labelled cytotoxin. The reaction may occur at room temperature or 37° C. Reactions times can occur between two days to several weeks. In one embodiment the reaction time is seven days to fourteen days.

In one embodiment, the cytotoxin is dissolved in an organic solvent, e.g., acetonitrile (ACN), prior to reacting with isotopically labeled water. In one embodiment, the cytotoxin in ACN is reacted with H₂¹⁸O to produce isotopically-labeled cytotoxin. In another embodiment, MMAF or MMAE in ACN is reacted with H₂¹⁸O to produce isotopically-labeled MMAF or MMAE. In a particular embodiment, the cytotoxin is reacted with isotopically-labeled water under strongly acidic conditions. The acid may include TFA or formic acid. In certain embodiments, isotopic purity of the labeled molecule can be assessed by ionizing and detecting the mass-to-charge ratios associated with isotopically-labeled cytotoxin.

In one embodiment, unoccupied cysteine sites of a cysteine-conjugated antibody drug conjugate (ADC) are conjugated with the isotopically-labeled cytotoxin.

In particular embodiments, a reductant is used to reduce the ADC inter-chain disulfide bonds to produce free sulfhydryl groups (e.g., unoccupied cysteine sites) prior to conjugation with the isotopically-labelled cytotoxin. The free sulfhydryl groups are then available for conjugation with the isotopically-labeled cytotoxin. Therefore, in one embodiment, the ADC is first reduced by the reductant and then conjugated with isotopically-labeled cytotoxin.

In one embodiment, the reductant is any compound or reagent that can reduce inter-chain disulfide bonds. In certain embodiments, the reductant is dithiothreitol (DTT), 2-mercaptoethanol, and/or tris(2-carboxyethyl)phosphine (TCEP). In a further embodiment, the reductant is DTT. In an alternative embodiment, the reductant is TCEP. Additional reducing agents can be employed in the methods disclosed herein and are well known by those skilled in the art.

Reductant is applied in order to reduce inter-chain disulfide bonds. In one embodiment, the reductant is applied in excess to ensure complete reduction of the inter-chain disulfide bonds in order to ensure subsequent labelling of most if not all disulfide bond sites. Methods for optimization of the amount of reducing agent are known to those skilled in the art. Concentrations of reductants may be added in increasing amounts until intra-chain disulfide bonds are reduced which can be detected by measuring separated heavy chains and light chains after the reduction reaction. If complete reduction of the inter-chain disulfide bonds does not occur, some of the disulfide bonds will not be labeled with cytotoxin (native or isotopically labelled) and may give an artificially higher quantification of native cytotoxin.

In one embodiment, excess reductant is removed prior to conjugation. In certain embodiments, reducing agent may interfere with the subsequently conjugation step, necessitating the remove of excess reductant prior to conjugation. For example, in one embodiment, excess reductant is removed by eluting the sample through a size exclusion chromatography column. In another embodiment, excess reductant is removed by molecular weight cut off (MWCO) filters. In a further embodiment, excess reductant is removed prior to conjugation with isotopically-labeled cytotoxin.

In one embodiment, conjugation occurs by reacting the ADC with the isotopically-labelled cytotoxin. Reaction may occur, for example, by mixing the ADC and isotopically-labeled cytotoxin at room temperature or at about 37° C. The reaction time can be optimized. In one embodiment, the reaction time is five minutes to about sixty minutes. In one embodiment, the isotopically labelled cytotoxin is isotopically labeled MMAF or MMAE. In one embodiment, the ratio of ADC to labelled cytotoxin is optimized to ensure that there are no disulfide bonds without cytotoxin (natural or isotopically labeled), e.g., all resulting ADC should have a drug load of 8 (DL8). Various methods for testing the ADC dug load are known by those skilled in the art.

In one embodiment, excess isotopically-labeled cytotoxin (not conjugated to the antibody) is removed prior to peptide mapping. In one embodiment, excess isotopically-labeled cytotoxin (not conjugated to the antibody) is removed prior to peptide mapping by eluting the sample through a size exclusion chromatography column.

In one embodiment, after conjugation, there results an ADC sample that has a mix of isotopically-labeled cytotoxin and non-isotopically-labeled cytotoxin (e.g. “natural cytotoxin”).

In another embodiment, subsequent to conjugation with isotopically-labeled cytotoxin, peptide mapping, e.g. via LC-MS/MS analysis, of the ADC sample is conducted. This step involves denaturing the isotopically-labeled antibody drug conjugate, reducing all remaining disulfide bonds, alkylating the resulting free sulfhydryls, enzymatically digesting the sample, and analyzing the sample by mass spectrometry. Various peptide mapping methods are well known to those skilled in the art and are described, for example, in Analytical Biochemistry 266, 31-47 (1999). In one embodiment, denaturing agents may include, for example, guanidine HCl, urea, or any denaturing agent that opens up all inter- and intra-disulfide bonds for subsequent reduction. Examples of reducing agents may include TCEP and DTT. After reduction, an alkylating agent can be applied to the sample to ensure that disulfide bonds do not re-form. An exemplary alkylating agent may include sodium iodoacetate. In certain embodiments, prior to enzymatic digesting, residual denaturing agent can be removed prior to the addition of the enzyme, e.g., by size exclusion chromatography. In certain embodiments, the peptides of the sample are enzymatically digested. Exemplary enzymes may include trypsin or Lys-C. Enzymatic digestion of the sample may be quenched by the addition of a strong acid, such as HCl or TFA. In some embodiments, the resulting peptides are then ionized, and the mass-to-charge ratios associated with naturally (non-isotopically labelled) and isotopically-labeled conjugated peptides are detected and compared.

### Antibody Drug Conjugates

The analysis methods described herein are particularly well suited for analyzing ADCs comprising cytotoxins containing a carbonyl group, e.g., MMAF or MMAE. In one embodiment the ADC is an anti-BCMA ADC. In a further embodiment, the anti-BCMA ADC is belantamab mafodotin. Belantamab mafodotin comprises an anti-BCMA antibody linked to a MMAF cytotoxic agent by a maleimidocaproyl (MC) linker.

The disclosure also provides compositions comprising anti-BCMA antibody drug conjugates (ADCs) and related methods for treating BCMA-mediated diseases or disorders. It will be understood that a composition comprising anti-BCMA ADCs, as described herein, may also be referred to as a population of anti-BCMA ADCs as described herein: the phrases being interchangeable.

In one embodiment, the anti-BCMA antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and/or a CDRL3 comprising the amino acid sequence according to SEQ ID NO:6.

In another embodiment, the anti-BCMA antibody comprises a heavy chain variable region (VH) comprising the amino acid sequence according to SEQ ID NO:7; and/or a light chain variable region (VL) comprising the amino acid sequence according to SEQ ID NO:8.

In yet another embodiment, the anti-BCMA antibody comprises a heavy chain (HC) comprising the amino acid sequence according to SEQ ID NO:9; and/or a light chain (LC) comprising the amino acid sequence according to SEQ ID NO:10.

In one aspect of the disclosure, the analytical methods described herein can determine the specific amino acid residue location of the cytotoxic agent on the antibody (e.g., belantamab) as well as the quantitative amount of the cytotoxin at each amino acid residue. In one embodiment, the cytotoxin is conjugated to cysteine containing amino acids, e.g., light chain (LC) C214, heavy chain (HC) C224, heavy chain hinge region (HC Hinge) C230, and/or heavy chain hinge region (HC Hinge) C233. In one embodiment, the heavy chain hinge region contains two cytotoxin molecules at both C230 and C233. This may be referred to herein as “HC Hinge DL2”. In another embodiment, the heavy chain hinge region contains one cytotoxin molecule at either C230 or C233. This may be referred to herein as “HC Hinge DL1”. The methods described herein are able to distinguish between HC Hinge DL2 and HC Hinge DL1 isoforms. When the HC Hinge DL1 isoforms are detected, the methods described herein are able to determine the presence or absence of the HC Hinge DL1 isoform.

In one embodiment the anti-BCMA antibody is belantamab comprising the heavy chain sequence of SEQ. ID. NO. 9 (CDRs are underlined; HC C224, HC C230, and HC C233 are in bold/underlined):

QVQLVQSGAEVKKPGSSVKVSCKASGGTFSNYWMHWVRQAPGQGLEWMG
ATYRGHSDTYYNQKFKGRVTITADKSTSTAYMELSSLRSEDTAVYYCAR
GAIYDGYDVLDNWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYT
QKSLSLSPGK

In one embodiment the anti-BCMA antibody is belantamab comprising the light chain sequence of SEQ. ID. NO. 10 (CDRs are underlined; LC C214 is in bold/underlined):

DIQMTQSPSSLSASVGDRVTITCSASQDISNYLNWYQQKPGKAPKLLIY
YTSNLHSGVPSRFSGSGSGTDFTLTISSLQPEDFATYYCQQYRKLPWTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

According to a further aspect of the disclosure, there is provided a composition comprising an anti-BCMA antibody (e.g., belantamab) conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the average drug-antibody ratio (DAR) is about 2.1 and the percentage drug load at LC C214 is between about 38% to about 44%, the percentage drug load at HC C224 is between about 40% to about 46%, the percentage drug load of HC hinge DL2 at HC C230 and C233 is between about 5% to about 9%, and/or the percentage drug load of HC hinge DL1 at HC C230 or HC C233 is between about 3% to about 7%.

Thus, according to a further aspect of the disclosure, there is provided a composition comprising an anti-BCMA antibody (e.g., belantamab) conjugated to a cytotoxic agent to form an antibody drug conjugate (ADC), wherein the antibody comprises a heavy chain amino acid sequence of SEQ ID NO:9 and a light chain amino acid sequence of SEQ ID NO:10; wherein the cytotoxic agent is MMAF or MMAE (in particular, MMAF); and wherein the average drug-antibody ratio (DAR) is about 2.1 and the percentage drug load at LC C214 is about 41%, the percentage drug load at HC C224 is about 43%, the percentage drug load of HC hinge DL2 at HC C230 and C233 is about 7%, and/or the percentage drug load of HC hinge DL1 at HC C230 or HC C233 is about 5%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the average drug-antibody ratio (DAR) is about 3.0 and the percentage drug load at LC C214 is between about 53% to about 59%, the percentage drug load at HC C224 is between about 55% to about 61%, the percentage drug load of HC hinge DL2 at HC C230 and C233 is between about 13% to about 19%, and/or the percentage drug load of HC hinge DL1 at HC C230 or HC C233 is between about 8% to about 14%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a heavy chain amino acid sequence of SEQ ID NO:9 and a light chain amino acid sequence of SEQ ID NO:10; wherein the cytotoxic agent is MMAF or MMAE (in particular, MMAF); and wherein the average drug-antibody ratio (DAR) is about 3.0 and the percentage drug load at LC C214 is about 56%, the percentage drug load at HC C224 is about 58%, the percentage drug load of HC hinge DL2 at HC C230 and C233 is about 16%, and/or the percentage drug load of HC hinge DL1 at HC C230 or HC C233 is about 11%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the average drug-antibody ratio (DAR) is about 3.5 and the percentage drug load at LC C214 is between about 60% to about 66%, the percentage drug load at HC C224 is between about 62% to about 68%, the percentage drug load of HC hinge DL2 at HC C230 and C233 is between about 20% to about 26%, and/or the percentage drug load of HC hinge DL1 at HC C230 or HC C233 is between about 8% to about 14%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a heavy chain amino acid sequence of SEQ ID NO:9 and a light chain amino acid sequence of SEQ ID NO:10; wherein the cytotoxic agent is MMAF or MMAE (in particular, MMAF); and wherein the average drug-antibody ratio (DAR) is about 3.5 and the percentage drug load at LC C214 is about 63%, the percentage drug load at HC C224 is about 65%, the percentage drug load of HC hinge DL2 at HC C230 and C233 is about 23%, and/or the percentage drug load of HC hinge DL1 at HC C230 or HC C233 is about 11%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the average drug-antibody ratio (DAR) is about 4.0 and the percentage drug load at LC C214 is between about 65% to about 71%, the percentage drug load at HC C224 is between about 68% to about 74%, the percentage drug load of HC hinge DL2 at HC C230 and C233 is between about 24% to about 30%, and/or the percentage drug load of HC hinge DL1 at HC C230 or HC C233 is between about 12% to about 18%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a heavy chain amino acid sequence of SEQ ID NO:9 and a light chain amino acid sequence of SEQ ID NO:10; wherein the cytotoxic agent is MMAF or MMAE (in particular, MMAF); and wherein the average drug-antibody ratio (DAR) is about 4.0 and the percentage drug load at LC C214 is about 68%, the percentage drug load at HC C224 is about 71%, the percentage drug load of HC hinge DL2 at HC C230 and C233 is about 27%, and/or the percentage drug load of HC hinge DL1 at HC C230 or HC C233 is about 15%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the average drug-antibody ratio (DAR) is about 4.6 and the percentage drug load at LC C214 is between about 72% to about 78%, the percentage drug load at HC C224 is between about 73% to about 79%, the percentage drug load of HC hinge DL2 at HC C230 and C233 is between about 37% to about 43%, and/or the percentage drug load of HC hinge DL1 at HC C230 or HC C233 is between about 13% to about 19%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a heavy chain amino acid sequence of SEQ ID NO:9 and a light chain amino acid sequence of SEQ ID NO:10; wherein the cytotoxic agent is MMAF or MMAE (in particular, MMAF); and wherein the average drug-antibody ratio (DAR) is about 4.6 and the percentage drug load at LC C214 is about 75%, the percentage drug load at HC C224 is about 76%, the percentage drug load of HC hinge DL2 at HC C230 and C233 is about 40%, and/or the percentage drug load of HC hinge DL1 at HC C230 or HC C233 is about 16%.

According to a further aspect of the disclosure, there is provided a composition comprising an anti-BCMA antibody conjugated to a cytotoxic agent to form an antibody drug conjugate (ADC), wherein the antibody (e.g., belantamab) comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the average drug-antibody ratio (DAR) is about 5.0 and the percentage drug load at LC C214 is between about 75% to about 81%, the percentage drug load at HC C224 is between about 77% to about 83%, the percentage drug load of HC hinge DL2 at HC C230 and C233 is between about 43% to about 49%, and/or the percentage drug load of HC hinge DL1 at HC C230 or HC C233 is between about 11% to about 17%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a heavy chain amino acid sequence of SEQ ID NO:9 and a light chain amino acid sequence of SEQ ID NO:10; wherein the cytotoxic agent is MMAF or MMAE (in particular, MMAF); and wherein the average drug-antibody ratio (DAR) is about 5.0 and the percentage drug load at LC C214 is about 78%, the percentage drug load at HC C224 is about 80%, the percentage drug load of HC hinge DL2 at HC C230 and C233 is about 46%, and/or the percentage drug load of HC hinge DL1 at HC C230 or HC C233 is about 14%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the average drug-antibody ratio (DAR) is about 5.7 and the percentage drug load at LC C214 is between about 81% to about 87%, the percentage drug load at HC C224 is between about 82% to about 88%, the percentage drug load of HC hinge DL2 at HC C230 and C233 is between about 56% to about 61%, and/or the percentage drug load of HC hinge DL1 at HC C230 or HC C233 is between about 10% to about 16%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a heavy chain amino acid sequence of SEQ ID NO:9 and a light chain amino acid sequence of SEQ ID NO:10; wherein the cytotoxic agent is MMAF or MMAE (in particular, MMAF); and wherein the average drug-antibody ratio (DAR) is about 5.7 and the percentage drug load at LC C214 is about 84%, the percentage drug load at HC C224 is about 85%, the percentage drug load of HC hinge DL2 at HC C230 and C233 is about 58%, and/or the percentage drug load of HC hinge DL1 at HC C230 or HC C233 is about 13%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the percentage drug load at LC C214 is between about 56% to about 80%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the percentage drug load at HC C224 is between about 58% to about 81%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the percentage drug load at of HC hinge DL2 at HC C230 and C233 is between about 15% to about 46%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the percentage drug load of HC hinge DL1 at HC C230 or HC C233 is between about 11% to about 15%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the percentage drug load at LC C214 is between about 56% to about 80%, the percentage drug load at HC C224 is between about 58% to about 81%, the percentage drug load of HC hinge DL2 at HC C230 and C233 is between about 15% to about 46%, and the percentage drug load of HC hinge DL1 at HC C230 or HC C233 is between about 11% to about 15%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the average drug-antibody ratio (DAR) is about 3 to about 5 and the percentage drug load at LC C214 is between about 56% to about 80%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the average drug-antibody ratio (DAR) is about 3 to about 5 and the percentage drug load at HC C224 is between about 58% to about 81%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the average drug-antibody ratio (DAR) is about 3 to about 5 and the percentage drug load at of HC hinge DL2 at HC C230 and C233 is between about 15% to about 46%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the average drug-antibody ratio (DAR) is about 3 to about 5 and the percentage drug load of HC hinge DL1 at HC C230 or HC C233 is between about 11% to about 15%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the average drug-antibody ratio (DAR) is about 3 to about 5 and the percentage drug load at LC C214 is between about 56% to about 80%, the percentage drug load at HC C224 is between about 58% to about 81%, the percentage drug load of HC hinge DL2 at HC C230 and C233 is between about 15% to about 46%, and the percentage drug load of HC hinge DL1 at HC C230 or HC C233 is between about 11% to about 15%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the percentage drug load at LC C214 is between about 63% to about 76%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the percentage drug load at HC C224 is between about 65% to about 78%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the percentage drug load of HC hinge DL2 at HC C230 and C233 is between about 22% to about 40%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the percentage drug load of HC hinge DL1 at HC C230 or HC C233 is between about 11% to about 16%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the percentage drug load at LC C214 is between about 63% to about 76%, the percentage drug load at HC C224 is between about 65% to about 78%, the percentage drug load of HC hinge DL2 at HC C230 and C233 is between about 22% to about 40%, and the percentage drug load of HC hinge DL1 at HC C230 or HC C233 is between about 11% to about 16%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the average drug-antibody ratio (DAR) is about 3.5 to about 4.6 and the percentage drug load at LC C214 is between about 63% to about 76%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the average drug-antibody ratio (DAR) is about 3.5 to about 4.6 and the percentage drug load at HC C224 is between about 65% to about 78%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the average drug-antibody ratio (DAR) is about 3.5 to about 4.6 and the percentage drug load of HC hinge DL2 at HC C230 and C233 is between about 22% to about 40%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the average drug-antibody ratio (DAR) is about 3.5 to about 4.6 and the percentage drug load of HC hinge DL1 at HC C230 or HC C233 is between about 11% to about 16%.

According to a further aspect of the disclosure, there is provided a composition comprising an anti-BCMA antibody (e.g., belantamab) conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the average drug-antibody ratio (DAR) is about 3.5 to about 4.6 and the percentage drug load at LC C214 is between about 63% to about 76%, the percentage drug load at HC C224 is between about 65% to about 78%, the percentage drug load of HC hinge DL2 at HC C230 and C233 is between about 22% to about 40%, and the percentage drug load of HC hinge DL1 at HC C230 or HC C233 is between about 11% to about 16%.

An anti-BCMA ADC in the compositions described herein may be useful in the treatment and/or prevention of various BCMA-mediated diseases, including, for example, B-cell mediated cancers such as lymphomas and multiple myeloma. An anti-BCMA ADC described herein may bind to human BCMA, for example, human BCMA containing the amino acid sequence of GenBank Accession Number Q02223.2 or BCMA proteins having at least 90% amino acid sequence homology or at least 90% amino acid sequence identity thereto.

The anti-BCMA ADC comprises an anti-BCMA antigen binding protein. The term “antigen binding protein” as used herein refers to antibodies, antibody fragments and other protein constructs which are capable of binding an antigen, for example an anti-BCMA antigen binding protein being capable of binding to BCMA, for example, human BCMA. An antigen binding protein may comprise heavy chain variable regions and light chain variable regions of the disclosure which may be formatted into the structure of a natural antibody or functional fragment or equivalent thereof. An antigen binding protein may therefore comprise the V_{H} regions of the disclosure formatted into a full-length antibody, a (Fab′)2 fragment, a Fab fragment, or equivalent thereof (such as scFV, bi- tri- or tetra-bodies, Tandabs etc.), when paired with an appropriate light chain. An antibody may be an IgG1, IgG2, IgG3, or IgG4; or IgM; IgA, IgE or IgD or a modified variant thereof. The constant domain of an antibody heavy chain may be selected accordingly. The light chain constant domain may be a kappa or lambda constant domain. Furthermore, an antigen binding protein may comprise modifications of all classes, e.g., IgG dimers, Fc mutants that no longer bind Fc receptors or mediate Clq binding. An antigen binding protein may also be a chimeric antibody of the type described in WO86/01533 which comprises an antigen binding region and a non-immunoglobulin region.

An antigen binding protein may be either a dAb, Fab, Fab′, F(ab′)2, Fv, diabody, triabody, tetrabody, miniantibody, or a minibody. An antigen binding protein may be either a fully human, a humanized, or a chimeric antibody. An antigen-binding protein may be an antibody that is humanized. An antigen-binding protein may be a monoclonal antibody

Exemplary anti-BCMA antigen binding proteins and methods of making the same are disclosed in International Publication No. WO2012/163805 which is incorporated by reference herein in its entirety. Additional exemplary anti-BCMA antigen binding proteins include those described in WO2016/014789, WO2016/090320, WO2016/090327, WO2016/020332, WO2016/079177, WO2014/122143, WO2014/122144, WO2017/021450, WO2016/014565, WO2014/068079, WO2015/166649, WO2015/158671, WO2015/052536, WO2014/140248, WO2013/072415, WO2013/072406, WO2014/089335, US2017/165373, WO2013/154760, and WO2017/051068, each of which is incorporated by reference herein in its entirety.

In another embodiment, an anti-BCMA antigen binding protein described herein may inhibit the binding of BAFF and/or APRIL to the BCMA receptor. In another embodiment, an anti-BCMA antigen binding protein described herein may be capable of binding to FcγRIIIA or is capable of FcγRIIIA mediated effector function.

An anti-BCMA antigen binding protein may comprise an antibody (“anti-BCMA antibody”). The term “antibody” as used herein refers to molecules with an immunoglobulin-like domain (e.g., IgG, IgM, IgA, IgD or IgE) and may include monoclonal, recombinant, polyclonal, chimeric, human, and humanized molecules of this type. Monoclonal antibodies may be produced by a eukaryotic cell clone or a prokaryotic close cell expressing an antibody. Monoclonal antibodies may also be produced by a eukaryotic cell line which can recombinantly express the heavy chain and light chain of the antibody by virtue of having nucleic acid sequences encoding these introduced into the cell. Exemplary methods for producing antibodies from different eukaryotic cell lines such as Chinese Hamster Ovary cells, hybridomas or immortalized antibody cells derived from an animal (e.g., human) are well known to those skilled in the art.

An antibody may be derived, for example, from either rat, mouse, primate (e.g., cynomolgus, Old World monkey or Great Ape), human, or other sources such as nucleic acids generated using molecular biology techniques known to those skilled in the art which encode an antibody molecule.

An antibody may comprise a constant region, which may be of any isotype or subclass. The constant region may be of the IgG isotype, for example, IgG₁, IgG₂, IgG₃, IgG₄ or variants thereof.

An antigen binding protein may comprise one or more modifications including, for example, a mutated constant domain such that, when the antigen binding protein is an antibody, the antibody has enhanced effector functions/ADCC and/or complement activation.

The anti-BCMA antibody may have enhanced antibody dependent cell mediated cytotoxic activity (ADCC) effector function. The term “effector function” as used herein is meant to refer to one or more of antibody-dependent cell-mediated cytotoxic activity (ADCC), complement-dependent cytotoxic activity (CDC) mediated responses, Fc-mediated phagocytosis and/or antibody recycling via the FcRn receptor. For IgG antibodies, effector functionalities may include ADCC and ADCP may be mediated by the interaction of the heavy chain constant region with a family of Fcγ receptors present on the surface of immune cells. In humans these may include FcγRI (CD64), FcγRII (CD32) and FcγRIII (CD16). Interaction between an antigen binding protein bound to antigen and the formation of the Fc/Fcγ complex may induce a range of effects including cytotoxicity, immune cell activation, phagocytosis and/or release of inflammatory cytokines.

The anti-BCMA antibody may inhibit the binding of BAFF and/or APRIL to BCMA receptor. The anti-BCMA antibody may be capable of binding to FcγRIIIA or may be capable of FcγRIIIA mediated effector function.

The anti-BCMA antibody may comprises two immunoglobulin (Ig) heavy chains (“HC”) and two Ig light chains (“LC”). The basic antibody structural unit may comprise, for example, a tetramer of subunits. Each tetramer may include two pairs of polypeptide chains, each pair having one “light” (about 25 kDa) and one “heavy” chain (about 50-70 kDa). The amino-terminal portion of each chain may include a variable region of about 100 to 110 or more amino acids primarily responsible for antigen recognition. This variable region may initially be expressed linked to a cleavable signal peptide. The variable region without the signal peptide may be referred to as a mature variable region. Thus, in one example, a light chain mature variable region may comprise a light chain variable region without the light chain signal peptide. The carboxy-terminal portion of each chain may define a constant region. The heavy chain constant region may be primarily responsible for effector function.

The mature variable regions of each light/heavy chain pair may form the antibody binding site (also referred to as the antigen binding site). “Antigen binding site” refers to a site on an antibody which is capable of specifically binding to an antigen, this may be a single variable domain, or it may be paired V_{H}NL domains as can be found on a standard antibody. Thus, an intact antibody may have, for example, two binding sites. Except in bifunctional or bispecific antibodies, the two binding sites can be the same. The chains all may exhibit the same general structure of relatively conserved framework regions (FR) joined by three hypervariable regions, also called complementarity determining regions or “CDRs”. The CDRs from the two chains of each pair may be aligned by the framework regions, enabling binding to a specific epitope. Thus, in one example, from N-terminal to C-terminal, both light and heavy chains comprise the domains FR1, CDR1, FR2, CDR2, FR3, CDR3 and FR4.

“CDRs” are defined as the complementarity determining region amino acid sequences of an antibody. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs. In one embodiment, a composition comprises an anti-BCMA antibody comprising one or more CDR's as described herein, or one or both of the heavy or light chain variable domains as described herein.

The terms “variant”, “antibody variant”, “CDR variant” and “post-translational modification variant” refers to at least one amino acid change in an antibody sequence. Variants may be the result of a post translational modification, a chemical change or a sequence change via at least one deletion, substitution or addition. Some post-translational modifications result in a chemical change which does not change the sequence (e.g., Met and oxidized Met; or Asp and isomerized/iso-Asp; or aggregation) while others result in a sequence change such as the conversion of one amino acid residue into another (e.g., Asn conversion to Asp via deamidation; or lysine deletion). Further post-translational modification variants are described below. A variant antibody sequence which comprises a sequence change may be the result of a designed sequence change or a post-translational modification. An amino acid sequence change may be a deletion, substitution or addition.

In one such embodiment, substitutions are conservative substitutions. In an alternative embodiment, an antibody variant comprises at least one substitution and retains the canonical structure of the antigen binding protein. In one embodiment, an antibody variant is at least about 80%, about 85%, about 90%, or about 95% identical to (e.g., has amino acid sequence identity to) the amino acid sequence of a parental antibody. In another embodiment, the antibody variant comprises a heavy chain amino acid sequence that is at least about 80%, about 85%, about 90%, or about 95% identical to the amino acid sequence of SEQ ID NO:9 and/or a heavy chain amino acid sequence that is at least about 80%, about 85%, about 90%, or about 95% identical to the amino acid sequence of SEQ ID NO:10.

Antigen binding proteins may have amino acid modifications (e.g., amino acid substitutions) that increase the affinity of the constant domain or fragment thereof for FcRn. Increasing the half-life (e.g., serum half-life) of therapeutic and diagnostic IgG antibodies and other bioactive molecules has many benefits including reducing the amount and/or frequency of dosing of these molecules. In one embodiment, an antigen binding protein of the disclosure comprises all or a portion (an FcRn binding portion) of an IgG constant domain having one or more of the following amino acid modifications.

For example, with reference to IgG1, M252Y/S254T/T256E (commonly referred to as “YTE”) and/or M428L/N434S (commonly referred to as “LS”) modifications increase FcRn binding at pH 6.0 (Wang et al. 2018).

Half-life can also be enhanced by T250Q/M428L, V2591N308F/M428L, N434A, and T307A/E380A/N434A modifications (with reference to IgG1 and Kabat numbering) (Monnet et al.).

Half-life and FcRn binding can also be extended by introducing H433K and N434F modifications (commonly referred to as “HN” or “NHance”) (with reference to IgG1) (WO2006/130834).

WO00/42072 discloses a polypeptide comprising a variant Fc region with altered FcRn binding affinity, which polypeptide comprises an amino acid modification at any one or more of amino acid positions 238, 252, 253, 254, 255, 256, 265, 272, 286, 288, 303, 305, 307, 309, 311, 312, 317, 340, 356, 360, 362, 376, 378, 380, 386,388, 400, 413, 415, 424, 433, 434, 435, 436, 439, and 447 of the Fc region (EU index numbering).

WO02/060919 discloses a modified IgG comprising an IgG constant domain comprising one or more amino acid modifications relative to a wild-type IgG constant domain, wherein the modified IgG has an increased half-life compared to the half-life of an IgG having the wild-type IgG constant domain, and wherein the one or more amino acid modifications are at one or more of positions 251, 253, 255, 285-290, 308-314, 385-389, and 428-435.

Shields et al. (2001, J Biol Chem; 276:6591-604) used alanine scanning mutagenesis to alter residues in the Fc region of a human IgG1 antibody and then assessed the binding to human FcRn. Positions that effectively abrogated binding to FcRn when changed to alanine include I253, S254, H435, and Y436. Other positions showed a less pronounced reduction in binding as follows: E233-G236, R255, K288, L309, S415, and H433. Several amino acid positions exhibited an improvement in FcRn binding when changed to alanine; notable among these are P238, T256, E272, V305, T307, Q311, D312, K317, D376, E380, E382, S424, and N434. Many other amino acid positions exhibited a slight improvement (D265, N286, V303, K360, Q362, and A378) or no change (S239, K246, K248, D249, M252, E258, T260, S267, H268, S269, D270, K274, N276, Y278, D280, V282, E283, H285, T289, K290, R292, E293, E294, Q295, Y296, N297, S298, R301, N315, E318, K320, K322, S324, K326, A327, P329, P331, E333, K334, T335, S337, K338, K340, Q342, R344, E345, Q345, Q347, R356, M358, T359, K360, N361, Y373, S375, S383, N384, Q386, E388, N389, N390, K392, L398, S400, D401, K414, R416, Q418, Q419, N421, V422, E430, T437, K439, S440, S442, S444, and K447) in FcRn binding.

The most pronounced effect with respect to improved FcRn binding was found for combination variants. At pH 6.0, the E380A/N434A variant showed over 8-fold better binding to FcRn, relative to native IgG1, compared with 2-fold for E380A and 3.5-fold for N434A. Adding T307A to this resulted in a 12-fold improvement in binding relative to native IgG1. In one embodiment, the antigen binding protein of the disclosure comprises E380A/N434A substitutions and has increased binding to FcRn.

Dall'Acqua et al. (2002, J Immunol.; 169:5171-80) describes random mutagenesis and screening of human IgG1 hinge-Fc fragment phage display libraries against mouse FcRn. They disclosed random mutagenesis of positions 251, 252, 254-256, 308, 309, 311, 312, 314, 385-387, 389, 428, 433, 434, and 436. The major improvements in IgG1-human FcRn complex stability occur when substituting residues located in a band across the Fc-FcRn interface (M252, S254, T256, H433, N434, and Y436) and to lesser extent substitutions of residues at the periphery, such as V308, L309, Q311, G385, Q386, P387, and N389. The variant with the highest affinity to human FcRn was obtained by combining the M252Y/S254T/T256E (“YTE”) and H433K/N434F/Y436H mutations and exhibited a 57-fold increase in affinity relative to the wild-type IgG1. The in vivo behavior of such a mutated human IgG1 exhibited a nearly 4-fold increase in serum half-life in cynomolgus monkey as compared to wild-type IgG1.

The antigen binding protein may have optimized binding to FcRn. Thus, the antigen binding protein may comprise at least one amino acid modification in the Fc region of said antigen binding protein, wherein said modification is at an amino acid position selected from the group consisting of: 226, 227, 228, 230, 231, 233, 234, 239, 241, 243, 246, 250, 252, 256, 259, 264, 265, 267, 269, 270, 276, 284, 285, 288, 289, 290, 291, 292, 294, 297, 298, 299, 301, 302, 303, 305, 307, 308, 309, 311, 315, 317, 320, 322, 325, 327, 330, 332, 334, 335, 338, 340, 342, 343, 345, 347, 350, 352, 354, 355, 356, 359, 360, 361, 362, 369, 370, 371, 375, 378, 380, 382, 384, 385, 386, 387, 389, 390, 392, 393, 394, 395, 396, 397, 398, 399, 400, 401 403, 404, 408, 411, 412, 414, 415, 416, 418, 419, 420, 421, 422, 424, 426, 428, 433, 434, 438, 439, 440, 443, 444, 445, 446 and 447 of the Fc region (numbering according to the EU index as in Kabat et al., Sequences of Proteins of Immunological Interest, 5th Ed. Public Health Service, National Institutes of Health, Bethesda, Md. (1991)).

Additionally, various publications describe methods for obtaining physiologically active molecules with modified half-lives, either by introducing an FcRn-binding polypeptide into the molecules (WO97/43316, U.S. Pat. Nos. 5,869,046, 5,747,035, WO96/32478 and WO91/14438) or by fusing the molecules with antibodies whose FcRn-binding affinities are preserved, but affinities for other Fc receptors have been greatly reduced (WO99/43713), or fusing with FcRn binding domains of antibodies (WO00/09560, U.S. Pat. No. 4,703,039).

FcRn affinity enhanced Fc variants to improve both antibody cytotoxicity and half-life were identified in screens at pH 6.0. The selected IgG variants can be produced as low fucosylated molecules. The resulting variants show increased serum persistence in hFcRn mice, as well as conserved enhanced ADCC (Monnet et al.) Exemplary variants include (with reference to IgG1 and numbering according to the EU index as in Kabat et al.):
P230T/V303A/K322R/N389T/F404L/N434S;
P228R/N434S;
Q311R/K334R/Q342E/N434Y;
C226G/Q386R/N434Y;
T307P/N389T/N434Y;
P230S/N434S;
P230T/V305A/T307A/A378V/L398P/N434S;
P23OT/P387S/N434S;
P230Q/E269D/N434S;
N276S/A378V/N434S;
T307A/N315D/A330V/382V/N389T/N434Y;
T256N/A378V/S383N/N434Y;
N315D/A330V/N361 D/A387V/N434Y;
V2591/N315D/M428L/N434Y;
P230S/N315D/M428L/N434Y;
F241 L/V264E/T307P/A378V/H433R;
T250A/N389K/N434Y;
V305A/N315D/A330V/P395A/N434Y;
V264E/Q386R/P396L/N434S/K439R;
E294del/T307P/N434Y (wherein ‘del’ indicates a deletion).

Also described is a method for the production of an antigen binding protein described herein comprising the steps of: a) culturing a recombinant host cell comprising an expression vector comprising the isolated nucleic acid as described herein, wherein the FUT8 gene encoding alpha-1,6-fucosyltransferase has been inactivated in the recombinant host cell; and b) recovering the antigen binding protein. Such methods for the production of antigen binding proteins can be performed, for example, using the POTELLIGENT technology system available from BioWa, Inc. (Princeton, NJ) in which CHOK1 SV cells lacking a functional copy of the FUT8 gene produce monoclonal antibodies having enhanced antibody dependent cell mediated cytotoxicity (ADCC) activity that is increased relative to an identical monoclonal antibody produced in a cell with a functional FUT8 gene. Aspects of the POTELLIGENT technology system are described in U.S. Pat. Nos. 7,214,775, 6,946,292, WO0061739 and WO0231240 all of which are incorporated herein by reference. Those of ordinary skill in the art will also recognize other appropriate systems and methods for generating antigen binding proteins, such as antibodies.

An antibody may be recovered and purified by conventional protein purification procedures. For example, the antibody may be harvested directly from the culture medium. Harvest of the cell culture medium may be via clarification, for example by centrifugation and/or depth filtration. Recovery of the antibody is followed by purification to ensure adequate purity. Therefore, also described is a cell culture medium comprising an antibody described herein. In one embodiment, the cell culture medium comprises CHO cells.

The antibody may be subsequently purified from the cell culture medium. This may comprise harvesting the cell culture supernatant, placing the cell culture supernatant in contact with a purification medium (e.g., protein A resin or protein G resin to bind antibody molecules) and eluting the antibody molecules from the purification medium to produce an eluate. Therefore, in one aspect, there is provided an eluate comprising an antibody described herein.

One or more chromatography steps may be used in purification, for example one or more chromatography resins; and/or one or more filtration steps. For example, affinity chromatography using resins, such as protein A, G, or L may be used to purify the composition. Alternatively, or in addition to, an ion-exchange resin such as a cation-exchange may be used to purify the composition.

Alternatively, the purification steps comprise an affinity chromatography resin step, followed by a cation-exchange resin step.

In one embodiment, an anti-BCMA antibody comprises a heavy chain variable region CDR1 (“CDRH1”) comprising an amino acid sequence with at least about 90%, 91%, 92%, 93%, 94%, 95%, 96%, 97%, 98%, 99% or 100% sequence identity to the amino acid sequence set forth in SEQ ID NO:1. In one embodiment, a heavy chain variable region CDR1 (“CDRH1”) comprises an amino acid sequence with one amino acid variation (“variant”) to the amino acid sequence set forth in SEQ ID NO:1.

In one embodiment, an anti-BCMA antibody comprises a heavy chain variable region CDR2 (“CDRH2”) comprising an amino acid sequence with at least about 90%, 91%, 92%, 93%, 94%, 95%, 96%, 97%, 98%, 99% or 100% sequence identity to the amino acid sequence set forth in SEQ ID NO:2. In one embodiment, a heavy chain variable region CDR2 (“CDRH2”) comprises an amino acid sequence with one amino acid variation (“variant”) to the amino acid sequence set forth in SEQ ID NO:2.

In one embodiment, an anti-BCMA antibody comprises a heavy chain variable region CDR3 (“CDRH3”) comprising an amino acid sequence with at least about 90%, 91%, 92%, 93%, 94%, 95%, 96%, 97%, 98%, 99% or 100% sequence identity to the amino acid sequence set forth in SEQ ID NO:3. In one embodiment, a heavy chain variable region CDR3 (“CDRH3”) comprises an amino acid sequence with one amino acid variation (“variant”) to the amino acid sequence set forth in SEQ ID NO:3.

In one embodiment, an anti-BCMA antibody comprises a light chain variable region CDR1 (“CDRL1”) comprising an amino acid sequence with at least about 90%, 91%, 92%, 93%, 94%, 95%, 96%, 97%, 98%, 99% or 100% sequence identity to the amino acid sequence set forth in SEQ ID NO:4. In one embodiment, a light chain variable region CDL1 (“CDR”) comprises an amino acid sequence with one amino acid variation (“variant”) to the amino acid sequence set forth in SEQ ID NO:4.

In one embodiment, the anti-BCMA antibody comprises a light chain variable region CDR2 (“CDRL2”) comprising an amino acid sequence with at least about 90%, 91%, 92%, 93%, 94%, 95%, 96%, 97%, 98%, 99% or 100% sequence identity to the amino acid sequence set forth in SEQ ID NO:5. In one embodiment, a light chain variable region CDL2 (“CDR2”) comprises an amino acid sequence with one amino acid variation (“variant”) to the amino acid sequence set forth in SEQ ID NO:5.

In one embodiment, the anti-BCMA antibody comprises a light chain variable region CDR3 (“CDRL3”) comprising an amino acid sequence with at least about 90%, 91%, 92%, 93%, 94%, 95%, 96%, 97%, 98%, 99% or 100% sequence identity to the amino acid sequence set forth in SEQ ID NO:6. In one embodiment, a light chain variable region CDL3 (“CDR3”) comprises an amino acid sequence with one amino acid variation (“variant”) to the amino acid sequence set forth in SEQ ID NO:6.

In one embodiment, the anti-BCMA antibody comprises a CDRH1 comprising an amino acid sequence with at least about 90%, 91%, 92%, 93%, 94%, 95%, 96%, 97%, 98%, 99% or 100% sequence identity to the amino acid sequence set forth in SEQ ID NO:1; a CDRH2 comprising an amino acid sequence with at least about 90%, 91%, 92%, 93%, 94%, 95%, 96%, 97%, 98%, 99% or 100% sequence identity to the amino acid sequence set forth in SEQ ID NO:2; a CDRH3 comprising an amino acid sequence with at least about 90%, 91%, 92%, 93%, 94%, 95%, 96%, 97%, 98%, 99% or 100% sequence identity to the amino acid sequence set forth in SEQ ID NO:3; a CDRL1 comprising an amino acid sequence with at least about 90%, 91%, 92%, 93%, 94%, 95%, 96%, 97%, 98%, 99% or 100% sequence identity to the amino acid sequence set forth in SEQ ID NO:4; a CDRL2 comprising an amino acid sequence with at least about 90%, 91%, 92%, 93%, 94%, 95%, 96%, 97%, 98%, 99% or 100% sequence identity to the amino acid sequence set forth in SEQ ID NO:5; and/or a CDRL3 comprising an amino acid sequence with at least about 90%, 91%, 92%, 93%, 94%, 95%, 96%, 97%, 98%, 99% or 100% sequence identity to the amino acid sequence set forth in SEQ ID NO:6.

In one embodiment, the anti-BCMA antibody comprises a heavy chain variable region (“V_{H}”) comprising an amino acid sequence with at least about 90%, 91%, 92%, 93%, 94%, 95%, 96%, 97%, 98%, 99% or 100% sequence identity to the amino acid sequence set forth in SEQ ID NO:7.

In one embodiment, the anti-BCMA antibody comprises a light chain variable region (“V_{L}”) comprising an amino acid sequence with at least about 90%, 91%, 92%, 93%, 94%, 95%, 96%, 97%, 98%, 99% or 100% sequence identity to the amino acid sequence set forth in SEQ ID NO:8.

In one embodiment, the anti-BCMA antibody comprises a V_{H} comprising an amino acid sequence with at least about 90%, 91%, 92%, 93%, 94%, 95%, 96%, 97%, 98%, 99% or 100% sequence identity to the amino acid sequence set forth in SEQ ID NO:7; and a V_{L} comprising an amino acid sequence with at least about 90%, 91%, 92%, 93%, 94%, 95%, 96%, 97%, 98%, 99% or 100% sequence identity to the amino acid sequence set forth in SEQ ID NO:8, wherein the anti-BCMA antibody retains binding to BCMA.

In one embodiment, the anti-BCMA antibody comprises a heavy chain region (“HC”) comprising an amino acid sequence with at least about 90%, 91%, 92%, 93%, 94%, 95%, 96%, 97%, 98%, 99% or 100% sequence identity to the amino acid sequence set forth in SEQ ID NO:9.

In one embodiment, the anti-BCMA antibody comprises a light chain region (“LC”) comprising an amino acid sequence with at least about 90%, 91%, 92%, 93%, 94%, 95%, 96%, 97%, 98%, 99% or 100% sequence identity to the amino acid sequence set forth in SEQ ID NO:10.

In one embodiment, the anti-BCMA antibody comprises a HC comprising an amino acid sequence with at least about 90%, 91%, 92%, 93%, 94%, 95%, 96%, 97%, 98%, 99% or 100% sequence identity to the amino acid sequence set forth in SEQ ID NO:9; and a LC comprising an amino acid sequence with at least about 90%, 91%, 92%, 93%, 94%, 95%, 96%, 97%, 98%, 99% or 100% sequence identity to the amino acid sequence set forth in SEQ ID NO:10.

“Percent identity” between a query amino acid sequence and a subject amino acid sequence is the “Identities” value, expressed as a percentage, that is calculated by the BLASTP algorithm when a subject amino acid sequence has 100% query coverage with a query amino acid sequence after a pair-wise BLASTP alignment is performed. Such pair wise BLASTP alignments between a query amino acid sequence and a subject amino acid

sequence are performed by using the default settings of the BLASTP algorithm available on the National Center for Biotechnology Institute's website with the filter for low complexity regions turned off. Importantly, a query sequence may be described by an amino acid sequence identified in one or more claims herein.

In one embodiment, an anti-BCMA antibody comprises a CDRH1 with the amino acid sequence set forth in SEQ ID NO:1; a CDRH2 with the amino acid sequence set forth in SEQ ID NO:2; a CDRH3 with the amino acid sequence set forth in SEQ ID NO:3; a CDRL1 with the amino acid sequence set forth in SEQ ID NO:4; a CDRL2 with the amino acid sequence set forth in SEQ ID NO:5; and a CDRL3 with the amino acid sequence set forth in SEQ ID NO:6.

In one embodiment, an anti-BCMA antibody comprises a V_{H} with the amino acid sequence set forth in SEQ ID NO:7; and a V_{L} with the amino acid sequence set forth in SEQ ID NO:8.

In one embodiment, the anti-BCMA antibody is belantamab comprising a HC with the amino acid sequence set forth in SEQ ID NO:9, and a LC with the amino acid sequence set forth in SEQ ID NO:10.

The sequences of antibodies can be determined by the Kabat numbering system (Kabat et al. Sequences of proteins of Immunological Interest NIH, 1987). Alternatively they can be determined using the Chothia numbering system (Al-Lazikani et al., (1997) JMB 273, 927-948), the contact definition method (MacCallum R. M., and Martin A. C. R. and Thornton J. M, (1996), Journal of Molecular Biology, 262 (5), 732-745) or any other established method for numbering the residues in an antibody and determining CDRs known to one skilled in the art. Other numbering conventions for antibody sequences available to a skilled person include “AbM” (University of Bath) and “contact” (University College London) methods. Lastly, antibody sequences can be sequentially numbered.

When numerical reference is made to an amino acid described herein, sequences may be numbered according to the Kabat method or to the sequential numbering method. Unless expressly stated otherwise, numerical reference to a specific amino acid number is described herein with sequential numbering system. Throughout this specification, the terms “CDR”, “CDRL1”, “CDRL2”, “CDRL3”, “CDRH1”, “CDRH2”, “CDRH3” follow Kabat numbering. The amino acid residues in the variable region sequences and full length antibody sequences are numbered sequentially to denote any antibody sequence variant position or post-translational modification variant position, such as an isomerized variant (e.g., D103), a deamidated variant (e.g. N388) or an oxidized variant (e.g., M34).

Reference to a position in the CDR (e.g., M34 or D103) provides the position number in relation to the entire antibody sequence (sequential numbering). Therefore, it will be understood that M34 of CDRH1 refers to the fourth residue of SEQ ID NO: 1, e.g. as underlined: NYWMH (SEQ ID NO: 1). Equally, D103 of CDRH3 refers to the fifth residue of SEQ ID NO: 3, e.g. as underlined: GAIYDGYDVLDN (SEQ ID NO: 3).

In one embodiment, the composition comprises an antibody variant comprising a change in one or more amino acids in the primary sequence. In one embodiment, a composition comprises an antibody that is at least about 90% identical to the heavy chain amino acid sequence of SEQ ID NO:9 and/or the light chain sequence of SEQ ID NO:10 with an amino acid change of aspartic acid (D) to asparagine (N), e.g., D103N at CDRH3 (e.g. D99N in Kabat numbering).

In another embodiment, a composition comprises an antibody comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, a CDRL3 with the amino acid sequence set forth in SEQ ID NO:6, and comprises an amino acid change of aspartic acid (D) to asparagine (N), e.g., D103N at CDRH3.

In another embodiment, an anti-BCMA antibody comprises belantamab and comprises an amino acid change of aspartic acid (D) to asparagine (N), e.g., D103N at CDRH3.

In one embodiment, a composition comprises a mixture of antibodies at least about 90% identical to the heavy chain amino acid sequence of SEQ ID NO:9 and/or the light chain sequence of SEQ ID NO:10, wherein about of ≥5%, ≥10%, ≥15%, ≥20%, ≥25%, ≥50%, ≥75%, or ≥90% of the antibody in the mixture comprises D103N at CDRH3.

In one embodiment, a composition comprises a mixture of antibodies comprising a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, a CDRL3 with the amino acid sequence set forth in SEQ ID NO:6, wherein about of ≥5%, ≥10%, ≥15%, ≥20%, ≥25%, ≥50%, ≥75%, or ≥90% of the antibody in the mixture comprises D103N at CDRH3.

In one embodiment, a composition comprises belantamab, wherein about of ≥5%, ≥10%, ≥15%, ≥20%, ≥25%, ≥50%, ≥75%, or ≥90% of belantamab comprises D103N at CDRH3.

In one embodiment, the composition comprises belantamab comprising at least one antibody variant using the Kabat numbering system selected from the group consisting of G27Y, S30T, A93T, A24G, K73T, M481, V67A, F71Y, D99N, M4L, and K45E.

### Antibody-Drug Conjugates

Antibody drug conjugates (ADCs) are an emerging class of potent anti-cancer agents, which have recently demonstrated remarkable clinical benefit. ADCs are comprised of a cytotoxic agent chemically bound to an antibody via a linker. Putatively, by a series of events, including antigen binding at the cell surface, endocytosis, trafficking to the lysosome, ADC degradation, release of payload, interruption of cellular processing (e.g., mitosis) and apoptosis, ADCs may destroy cancer cells possessing an over-expression of cell-surface proteins. ADCs combine the antigen-driven targeting properties of monoclonal antibodies with the potent anti-tumor effects of cytotoxic agents. For example, in 2011 ADCETRIS® (an anti-CD30 antibody-MMAE ADC) gained regulatory approval for the treatment of refractory Hodgkin lymphoma and systemic anaplastic lymphoma.

ADCs have been used for the local delivery of cytotoxic agents, e.g., drugs that kill or inhibit the growth or proliferation of cells, in the treatment of cancer (Lambert, J. (2005) Curr. Opinion in Pharmacology 5:543-549; Wu et al. (2005) Nature Biotechnology 23(9):1137-1146; Payne, G. (2003) i 3:207-212; Syrigos and Epenetos (1999) Anticancer Research 19:605-614; Niculescu-Duvaz and Springer (1997) Adv. Drug Deliv. Rev. 26:151-172; U.S. Pat. No. 4,975,278). ADCs allow for the targeted delivery of a drug moiety to a tumor, and intracellular accumulation therein, where systemic administration of unconjugated drugs may result in unacceptable levels of toxicity to normal cells as well as the tumor cells sought to be eliminated (Baldwin et al., Lancet (Mar. 15, 1986) pp. 603-05; Thorpe (1985) “Antibody Carriers Of Cytotoxic Agents In Cancer Therapy: A Review,” in Monoclonal Antibodies '84: Biological And Clinical Applications (A. Pinchera et al., eds) pp. 475-506. Both polyclonal antibodies and monoclonal antibodies have been reported as useful in these strategies (Rowland et al., (1986) Cancer Immunol. Immunother. 21:183-87). Toxins used in antibody-toxin conjugates include bacterial toxins such as diphtheria toxin, plant toxins such as ricin, small molecule toxins such as geldanamycin (Mandler et al (2000) J. Nat. Cancer Inst. 92(19):1573-1581; Mandler et al. (2000) Bioorganic & Med. Chem. Letters 10:1025-1028; Mandler et al (2002) Bioconjugate Chem. 13:786-791), maytansinoids (EP 1391213; Liu et al. (1996) Proc. Natl. Acad. Sci. USA 93:8618-8623), and calicheamicin (Lode et al (1998) Cancer Res. 58:2928; Hinman et al. (1993) Cancer Res. 53:3336-3342).

In one embodiment, the anti-BCMA ADC comprises an antibody or antibody fragment conjugated to one or more cytotoxic agents, such as a chemotherapeutic agent, a drug, a growth inhibitory agent, a toxin (e.g., a protein toxin, an enzymatically active toxin of bacterial, fungal, plant, or animal origin, or fragments thereof), or a radioactive isotope (e.g., a radioconjugate).

In one embodiment, an anti-BCMA ADC has the following general structure:

ABP-((Linker)ₙ-Ctx)ₘ

Wherein:
ABP is an antigen binding protein, antibody, or antibody fragment;
Linker is either absent or any a cleavable or non-cleavable linker;
Ctx is any cytotoxic agent described herein;
n is 0, 1, 2, or 3; and,
m is 1, 2, 3, 4, 5, 6, 7, 8, 9 or 10.

In exemplary embodiments, enzymatically active toxins and fragments thereof that could be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, or the tricothecenes. See, e.g., WO 93/21232 published Oct. 28, 1993. A variety of radionuclides are available for the production of radio-conjugated antibodies, including, e.g., ²¹¹At, ²¹²Bi, ¹³¹I, ¹³¹In, ⁹⁰Y, or ¹⁸⁶Re.

An anti-BCMA antibody or fragment thereof of the present disclosure may also be conjugated to one or more cytotoxic agents, including, but not limited to, a calicheamicin, maytansinoids, dolastatins, auristatins, a trichothecene, and CC1065, or derivatives of these toxins that have toxin activity. Suitable cytotoxic agents include, for example, an auristatin including monomethyl auristatin (MMAF) and monomethyl auristatin E (MMAE) as well as ester forms of MMAE, a DNA minor groove binding agent, a DNA minor groove alkylating agent, an enediyne, a lexitropsin, a duocarmycin, a taxane, including paclitaxel and docetaxel, a puromycin, a dolastatin, a maytansinoid, and a vinca alkaloid. Specific cytotoxic agents include topotecan, morpholino-doxorubicin, rhizoxin, cyanomorpholino-doxorubicin, dolastatin-10, echinomycin, combretatstatin, chalicheamicin, maytansine, DM-1, DM-4, netropsin. Other suitable cytotoxic agents include anti-tubulin agents, such as an auristatin, a vinca alkaloid, a podophyllotoxin, a taxane, a baccatin derivative, a cryptophysin, a maytansinoid, a combretastatin, or a dolastatin. Antitubulin agent include dimethylvaline-valinedolaisoleuine-dolaproine-phenylalanine-p-phenylened-iamine (AFP), vincristine, vinblastine, vindesine, vinorelbine, VP-16, camptothecin, paclitaxel, docetaxel, epothilone A, epothilone B, nocodazole, colchicines, colcimid, estramustine, cemadotin, discodermolide, maytansine, DM-1, DM-4 or eleutherobin.

In one embodiment, an anti-BCMA ADC comprises an anti-BCMA antibody linked to MMAE or MMAF.

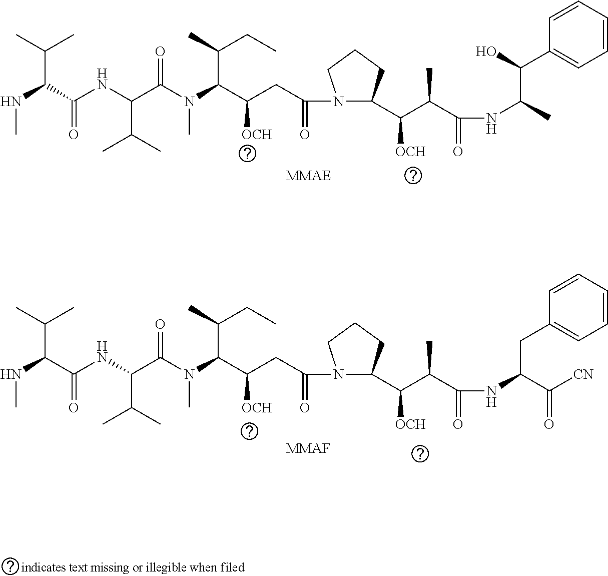

Exemplary linkers include cleavable and non-cleavable linkers. A cleavable linker may be susceptible to cleavage under intracellular conditions. Suitable cleavable linkers include, for example, a peptide linker cleavable by an intracellular protease, such as lysosomal protease or an endosomal protease. In exemplary embodiments, the linker can be a dipeptide linker, such as a valine-citrulline (val-cit) or a phenylalanine-lysine (phe-lys) linker. Other suitable linkers include, for example, linkers hydrolyzable at a pH of less than 5.5, such as a hydrazone linker. Additional suitable cleavable linkers include, for example, disulfide linkers. Exemplary linkers include 6-maleimidocaproyl (MC), maleimidopropanoyl (MP), valine-citrulline (val-cit), alanine-phenylalanine (ala-phe), p-aminobenzyloxycarbonyl (PAB), N-succinimidyl 4-(2-pyridylthio)pentanoate (SPP), N-succinimidyl 4-(N-maleimidomethyl)cyclohexane-1 carboxylate (SMCC), and N-succinimidyl (4-iodo-acetyl) aminobenzoate (SIAB).

In one embodiment, a linker may comprise of a thiol-reactive maleimide, a caproyl spacer, dipeptide valine-5 citrulline, a p-aminobenzyloxycarbonyl, a self-immolative fragmenting group, or a protease-resistant maleimidocaproyl.

In another embodiment, an anti-BCMAADC comprises an anti-BCMA antibody linked to MMAE or MMAF by an MC linker as depicted in the following structures:

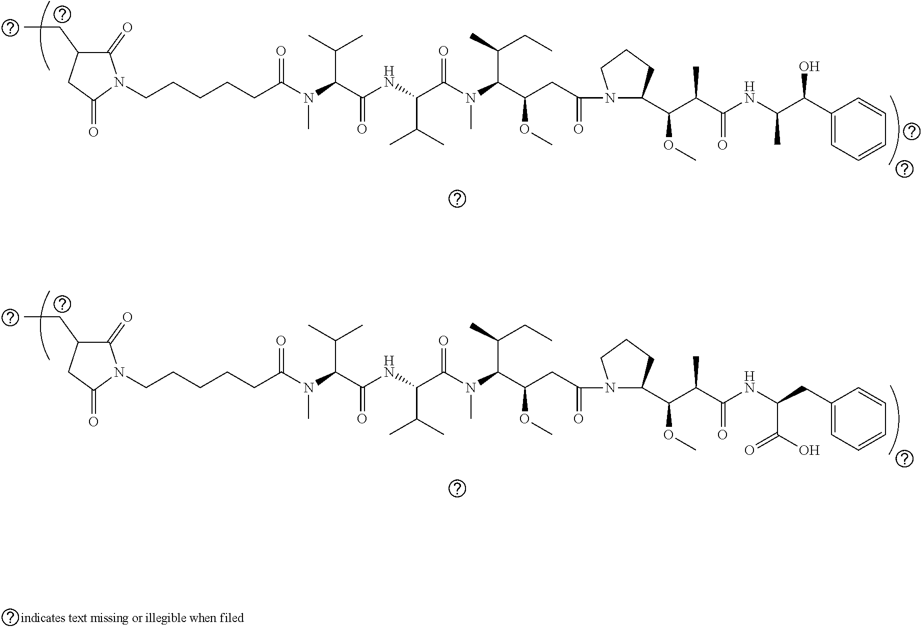 [SMILES: CCC1CC(=O)N(CCCCCC(=O)N(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@H](C)[C@@H](O)c2ccccc2)OC)C(C)C)C(C)C)C1=O ; CCC1CC(=O)N(CCCCCC(=O)N(C)[C@H](C(=O)N[C@H](C(=O)N(C)[C@@H]([C@@H](C)CC)[C@@H](CC(=O)N2CCC[C@H]2[C@H](OC)[C@@H](C)C(=O)N[C@@H](Cc2ccccc2)C(=O)O)OC)C(C)C)C(C)C)C1=O]

An anti-BCMA ADC described herein may contain any anti-BCMA antibody described herein with any cytotoxic agent described herein.

In one embodiment, an anti-BCMA ADC comprises an anti-BCMA antibody comprising a CDRH1 comprising an amino acid sequence with at least about 90%, 91%, 92%, 93%, 94%, 95%, 96%, 97%, 98%, 99% or 100% sequence identity to the amino acid sequence set forth in SEQ ID NO:1; a CDRH2 comprising an amino acid sequence with at least about 90%, 91%, 92%, 93%, 94%, 95%, 96%, 97%, 98%, 99% or 100% sequence identity to the amino acid sequence set forth in SEQ ID NO:2; a CDRH3 comprising an amino acid sequence with at least about 90%, 91%, 92%, 93%, 94%, 95%, 96%, 97%, 98%, 99% or 100% sequence identity to the amino acid sequence set forth in SEQ ID NO:3; a CDRL1 comprising an amino acid sequence with at least about 90%, 91%, 92%, 93%, 94%, 95%, 96%, 97%, 98%, 99% or 100% sequence identity to the amino acid sequence set forth in SEQ ID NO:4; a CDRL2 comprising an amino acid sequence with at least about 90%, 91%, 92%, 93%, 94%, 95%, 96%, 97%, 98%, 99% or 100% sequence identity to the amino acid sequence set forth in SEQ ID NO:5; and/or a CDRL3 comprising an amino acid sequence with at least about 90%, 91%, 92%, 93%, 94%, 95%, 96%, 97%, 98%, 99% or 100% sequence identity to the amino acid sequence set forth in SEQ ID NO:6; and is conjugated to MMAE or MMAF.

In yet another embodiment, an anti-BCMA ADC comprises an anti-BCMA antibody comprising a CDRH1 with the amino acid sequence set forth in SEQ ID NO:1; a CDRH2 with the amino acid sequence set forth in SEQ ID NO:2; a CDRH3 with the amino acid sequence set forth in SEQ ID NO:3; a CDRL1 with the amino acid sequence set forth in SEQ ID NO:4; a CDRL2 with the amino acid sequence set forth in SEQ ID NO:5; and a CDRL3 with the amino acid sequence set forth in SEQ ID NO:6; and is conjugated to MMAF or MMAE.

In one embodiment, an anti-BCMA ADC comprises an anti-BCMA antibody comprising a V_{H} comprising an amino acid sequence with at least about 90%, 91%, 92%, 93%, 94%, 95%, 96%, 97%, 98%, 99% or 100% sequence identity to the amino acid sequence set forth in SEQ ID NO:7; and/or a V_{L} comprising an amino acid sequence with at least about 90%, 91%, 92%, 93%, 94%, 95%, 96%, 97%, 98%, 99% or 100% sequence identity to the amino acid sequence set forth in SEQ ID NO:8; and is conjugated to MMAE or MMAF.

In yet another embodiment, an anti-BCMA ADC comprises an anti-BCMA antibody comprising a V_{H} with the amino acid sequence set forth in SEQ ID NO:7; and a V_{L} with the amino acid sequence set forth in SEQ ID NO:8; and is conjugated to MMAF or MMAE.

In one embodiment, an anti-BCMA ADC comprises an anti-BCMA antibody comprising a HC comprising an amino acid sequence with at least about 90%, 91%, 92%, 93%, 94%, 95%, 96%, 97%, 98%, 99% or 100% sequence identity to the amino acid sequence set forth in SEQ ID NO:9; and/or a LC comprising an amino acid sequence with at least about 90%, 91%, 92%, 93%, 94%, 95%, 96%, 97%, 98%, 99% or 100% sequence identity to the amino acid sequence set forth in SEQ ID NO:10; and is conjugated to MMAF or MMAE.

In yet another embodiment, an anti-BCMA ADC is belantamab mafodotin comprising an anti-BCMA antibody comprising a HC with the amino acid sequence set forth in SEQ ID NO:9, and a LC with the amino acid sequence set forth in SEQ ID NO:10; and is conjugated to MMAF.

### Preparation and Characterization of ADCs

Certain natural IgG1 molecules comprise 16 disulfide bonds (32 cysteines or sulfhydryl groups). In certain aspects, an antibody can be reduced in such a way that only the four interchain disulfide bonds are reduced and conjugated to a cytotoxic agent, allowing for up to eight sites of attachment for the cytotoxic agent. In other words, the drug load (“DL”), e.g. number of cytotoxic agents per antibody molecule can range from 0 to 8 and are described herein as DL0, DL2 (including DL2a and DL2b), DL4 (including DL4a, DL4b, and DL4c), DL6 (including DL6a and DL6b), and DL8.

The conjugation process may lead to heterogeneity in drug-antibody attachment for a given ADC composition, varying in both 1) the number of drugs bound to each antibody molecule and 2) the location of the cytotoxic agent. This may lead to an ADC composition with various DL species. The average drug-antibody ratio of the entire heterogenous ADC composition is referred to herein as “average DAR” or “DAR”. For example, an ADC composition may comprise of mixture of antibody species each with their own DL (some species in the mixture are DL2, some species in the mixture are DL4, some species in the mixture are DL6, and some species in the mixture are DL8) and the average DAR for the entire composition may be about 4.

In another embodiment, the term “percent DL” may be used to describe the percent of a specific DL species within the heterogenous ADC composition (e.g., percent DL2 is about 10% to about 30% of the total heterogenous ADC composition).

Drugs may be conjugated to antibodies via sulfhydryl groups on the antibody. The sulfhydryl groups can be sulfhydryl groups on cysteine side chains. The cysteine residues can be naturally present in an antibody (e.g., interchain disulfides) or introduced by other means, e.g., mutagenesis. Methods of conjugating drugs to sulfhydryl groups on antibodies are well-known in the art (see, e.g., U.S. Pat. Nos. 7,659,241, 7,498,298, and International Publication No. WO 2011/130613, WO 2014/152199, WO 2015/077605 and Bioconjugate Chem. 2005, 16, 1282-1290). Antibodies are typically reduced prior to conjugation in order to render sulfhydryl groups available for conjugation. Antibodies can be reduced using known conditions in the art. Reducing conditions are those that generally do not cause any substantial denaturation of the antibody and generally do not affect the antigen binding affinity of the antibody.

The reducing agent used in the reduction step may be TCEP and the TCEP may be added, e.g., at an excess for thirty minutes at room temperature. For example, 250 μL of a 10 mM solution of TCEP at pH 7.4 will readily reduce the interchain disulfides of 1 to 100 μg of antibody in thirty minutes at room temperature. Other reducing agents and conditions, however, can be used. Examples of reaction conditions include temperatures from 5° C. to 37° C. over a pH range of 5 to 8.

Various methods exist, and are known to those skilled in the art, for calculating the percent DL species and/or average DAR in an ADC composition. For example, heterogeneity of cysteine-linked ADCs is typically measured by hydrophobic interaction chromatography (HIC) which separates DL species based on the number of drugs loaded. LC-MS assays have also been developed to assess DL distribution. Exemplary methods for calculating the drug load distribution in an ADC composition can be found, for example, in Journal of Chromatography B 1060 (2017) 182-189.

For example, DL0 has no drug load on the antibody. For example, DL2 has a drug load of two. In one embodiment, the conjugation sites for DL2 are LC C214 and HC C224. For example, DL4 has a drug load of four. In one embodiment, the conjugation sites for DL4a are LC C214, HC C224, LC C214 and HC C224. In one embodiment, the conjugation sites for DL4b are HC C230, HC C233, HC C230 and HC C233. For example, DL6 has a drug load of six. In one embodiment, the conjugation sites for DL6 are LC C214, HC C224, HC C230, HC C233, HC C230 and HC C233. For example, DL8 has a drug load of eight. In one embodiment, the conjugation sites for DL8 are LC C214, HC C224, LC C214, HC C224, HC C230, HC C233, HC C230 and HC C233.

In one embodiment, the percent of a specific DL species (e.g., percent DL0, percent DL2, percent DL4a, percent DL4b, percent DL6, percent DL8) may be determined by separating individual DL species using hydrophobic interaction chromatography (HIC), calculating the area under the curve for each DL peak, and dividing each DL peak by the total area under the curve for all DL species combined. In one embodiment, the average DAR can be calculated from the area under the curve of each DL species using the following formula:

$$\% DL {Component}_{x} = \frac{{A}_{x}}{{A}_{0} + {A}_{1} + {A}_{2} + {A}_{3} + {A}_{4 a} + {A}_{4 b} + {A}_{5} + {A}_{6} + {A}_{8} + {A}_{10}} \times 100$$

$$DAR = \frac{( {A}_{1} \times 2 ) + ( {A}_{2} \times 2 ) + ( {A}_{3} \times 2 ) + ( {A}_{4 a} \times 4 ) + ( {A}_{4 b} \times 4 ) + ( {A}_{5} \times 6 ) + ( {A}_{6} \times 6 ) + ( {A}_{8} \times 8 ) + ( {A}_{10} \times 8 )}{{A}_{0} + {A}_{1} + {A}_{2} + {A}_{3} + {A}_{4 a} + {A}_{4 b} + {A}_{5} + {A}_{6} + {A}_{8} + {A}_{10}}$$

$$Where :$$

$${A}_{x} = \qquad Peak area of loading \times peak$$

$$X = \qquad {A}_{0} , {A}_{1} , {A}_{2} , {A}_{3} , {A}_{4 a} , {A}_{4 b} , {A}_{5} , {A}_{6} , {A}_{8} , and {A}_{10}$$

$${A}_{0} , {A}_{1} , {A}_{2} , {A}_{3} , {A}_{4 a} , {A}_{4 b} , {A}_{5} , {A}_{6} , {A}_{8} , and {A}_{10} = \qquad is the peak area of DL 0 , DL 1 , DL 2 , DL 3 , DL 4 a , DL 4 b , DL 5 , DL 6 , DL 8 and DL 10 peaks ( \text{} only \text{ } including peaks \geq ( 0.08 \% ) )$$

In one embodiment, the percent of a specific DAR sub-species (e.g., percent of DL2a in total DL2) is determined by collection of a specific DL species using a combination of analytical techniques that could include HIC, non-reducing separation methods, and mass spectrometric techniques.

In one embodiment, an anti-BCMA ADC composition has an average DAR of about 2 to about 7, about 2 to about 6, about 2.1 to about 5.7, about 2.1 to about 5.0, about 2.1 to about 4.6, about 2.1 to about 4.1, about 2.1 to about 3.5, about 2.1 to about 3.0, about 3.0 to about 5.7, about 3.0 to about 5.0, about 3.0 to about 4.6, about 3.0 to about 4.1, about 3.0 to about 3.5, about 3.5 to about 5.7, about 3.5 to about 5.0, about 3.5 to about 4.6, about 3.5 to about 4.1, about 3.8 to about 4.5, about 4.1 to about 5.7, about 4.1 to about 5.0, about 4.1 to about 4.6, about 4.6 to about 5.7, about 4.6 to about 5.0, about 5.0 to about 5.7, about 2.1, about 3.0, about 3.5, about 4.1, about 4.6, about 5.0, or about 5.7

In another embodiment, a composition comprises an anti-BCMA ADC, wherein the average DAR is about 2.1 to about 5.7, about 3.4 to about 4.6, about 3.8 to about 4.5, or about 4.

In one embodiment, a composition comprises an anti-BCMA ADC, wherein the antibody comprises a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; wherein the cytotoxic agent is MMAE or MMAF; and wherein the average DAR is about 2 to about 6, about 2.1 to about 5.7, about 3.4 to about 4.6, or about 3.8 to about 4.5.

In one embodiment, a composition comprises an anti-BCMA ADC, wherein the antibody comprises a V_{H} with the amino acid sequence set forth in SEQ ID NO:7, and a V_{L} with the amino acid sequence set forth in SEQ ID NO:8; wherein the cytotoxic agent is MMAE or MMAF; and wherein the average DAR is about 2 to about 6, about 2.1 to about 5.7, about 3.4 to about 4.6, or about 3.8 to about 4.5.

In one embodiment, the composition comprises belantamab mafodotin, wherein the average DAR is about 2 to about 6, about 2.1 to about 5.7, about 3.4 to about 4.6, or about 3.8 to about 4.5.

In one embodiment, percent DL0 species in an anti-BCMA ADC composition is about 10% or less, about 5% or less, about 1% to about 10%, about 1% to about 5%, or about 2.8% to about 4.7%.

In one embodiment, percent DL2 species in an anti-BCMA ADC composition is at least about 10%, at least about 15%, about 15.8% to about 26.3%, about 15% to about 27%, about 15% to about 32%, or about 10% to about 40%.

In one embodiment, percent DL4a species in an anti-BCMA ADC composition is at least about 30%, at least about 35%, about 35.5% to about 37.9%, about 35% to about 38%, about 30% to about 40%, or about 20% to about 50%. In another embodiment, percent DL4a species is the predominant species in the anti-BCMA ADC composition and comprises about ≥30%, ≥40%, ≥50%, ≥60%, ≥70%, ≥80%, or ≥90% of the all species combined.

In one embodiment, percent DL4b species in an anti-BCMA ADC composition is at least about 5%, at least about 7%, about 7.1% to about 8.5%, about 7% to about 9%, about 5% to about 10%, or about 1% to about 15%.

In one embodiment, percent DL6 species in an anti-BCMA ADC composition is at least about 10%, at least about 14%, about 14.0% to about 19.1%, about 14% to about 20%, about 10% to about 20%, or about 5% to about 30%.

In one embodiment, percent DL8 species in an anti-BCMA ADC composition is at least about 1%, at least about 6%, about 6.0% to about 12.0%, about 4% to about 15%, or about 1% to about 20%.

In one embodiment, a composition comprises an anti-BCMA ADC, wherein percent DL2 is about 15% to about 27% or about 15% to about 32%, percent DL4a is about 35% to about 38% or about 30% to about 40%, percent DL4b is about 7% to about 9% or about 5% to about 10%, percent DL6 is about 14% to about 20% or about 10% to about 20%, and/or DL8 is about 6.0% to about 12.0% or about 4% to about 15%.

In one embodiment, a composition comprises belantamab mafodotin, wherein percent DL2 is about 15% to about 27% or about 15% to about 32%, percent DL4a is about 35% to about 38% or about 30% to about 40%, percent DL4b is about 7% to about 9% or about 5% to about 10%, percent DL6 is about 14% to about 20% or about 10% to about 20%, and/or DL8 is about 6.0% to about 12.0% or about 4% to about 15%.

The term “undesired DAR species”, as used herein, refers to any DAR species which is not desired in the final composition and which may have a negative impact on certain properties (e.g., target binding, efficacy, safety, etc.) of the final therapeutic product. In one embodiment, an undesired DAR species is DL0 e.g., antibody not bound with cytotoxic agent after the conjugation process. In one embodiment, percent DL0 in the ADC composition is less than or equal to about 15%, about 14%, about 13%, about 12%, about 11%, about 10%, about 9%, about 8%, about 7%, about 6%, about 5%, about 4%, about 3%, about 2%, about 1%, or about 0.5%. In another embodiment, percent DL0 in the ADC composition is about 1% to about 10%, about 2% to about 5%, or about 2.0% to about 4.8%.

### Post-Translational Modifications

A “post-translational modification product” of an antibody described herein is an antibody composition wherein all or a portion of the composition comprises a “post-translational modification”. Post-translational modifications are changes to the antibody that may be the result from production of the antibody in a host cell, upstream and downstream manufacture, and/or storage (e.g., effect of exposure to light, temperature, pH, water, or by reaction with an excipient and/or the immediate container closure system). Therefore, the composition of the disclosure may be formed from the manufacture or storage of the antibody. Exemplary post-translational modifications comprise antibody sequence changes (“antibody variant” as described above), cleavage of certain leader sequences, the addition of various sugar moieties in various glycosylation patterns, non-enzymatic glycation, deamidation, oxidation, disulfide bond scrambling and other cysteine variants such as free sulfhydryls, racemized disulfides, thioethers and trisulfide bonds, isomerization, C-terminal lysine cleavage, and/or N-terminal glutamine cyclization.

In one example, a post-translational modification product comprises a “product-related impurity” that comprises a chemical change that results in reduced function and/or activity. In another example, a post-translational modification product comprises a “product-related substance” that comprises a chemical change that does not result in reduced function and/or activity. Product related impurities for the antibodies described herein include isomerized variants and oxidized variants. Product related substances for the antibodies described herein include deamidated variants, glycosylation variants, C-terminal cleaved variants and N-terminal pyro-glutamate variants.

In one embodiment, the composition comprises a heavy chain sequence of SEQ ID NO:9, and a light chain sequence of SEQ ID NO:10, comprising one or more functional post-translational modifications thereof. In another embodiment, the composition comprises a heavy chain sequence of SEQ ID NO:11, SEQ ID NO:12, SEQ ID NO:13, or SEQ ID NO:14, and a light chain of SEQ ID NO:10, comprising one or more functional post-translational modifications thereof.

The percent variant provided herein is expressed as a percentage of the total amount of antibody in the composition (e.g., a “population” of antibodies). For example, 40% or less oxidized variant refers to a total amount of 100% antibody in the composition of which 40% or less is oxidized. For example, 25% or less isomerized variant refers to a total amount of 100% antibody in the composition of which 25% or less is isomerized.

Glycation is a post-translational modification comprising a non-enzymatic chemical reaction between a reducing sugar, such as glucose, and a free amine group in the protein, and is typically observed at the epsilon amine of lysine side chains or at the N-terminus of the protein. Glycation can occur during production and/or storage in the presence of reducing sugars.

Deamidation, which may, for example, occur during production and/or storage, may be an enzymatic reaction or a chemical reaction. Deamidation may occur via simple chemical reaction through intramolecular cyclization where the amide nitrogen of the next amino acid in the chain nucleophilicly attacks the amide(N+1 attacks N) forming a succinimide intermediate. Deamidation may primarily convert asparagine (N) to iso-aspartic acid (iso-aspartate) and aspartic acid (aspartate) (D) at an approximately 3:1 ratio. This deamidation reaction may therefore be related to isomerization of aspartate (D) to iso-aspartate. The deamidation of asparagine and the isomerization of aspartate, both may involve the intermediate succinimide. To a much lesser degree, deamidation can occur with glutamine residues in a similar manner. Deamidation can occur in a CDR, in a Fab (non-CDR region), or in an Fc region. Isomerization is the conversion of aspartate (D) to iso-aspartate which involves the intermediate succinimide.

Oxidation can occur during production and/or storage (e.g., in the presence of oxidizing conditions) and results in a covalent modification of a protein, induced either directly by reactive oxygen species or indirectly by reaction with secondary by-products of oxidative stress. Oxidation may happen primarily with methionine residues, but may also occur at tryptophan and free cysteine residues. Oxidation can occur in a CDR, in a Fab (non-CDR) region, or in an Fc region.

Disulfide bond scrambling can occur during production and/or storage conditions. Under certain circumstances, disulfide bonds may break or form incorrectly, resulting in unpaired cysteine residues (—SH). These free (unpaired) sulfhydryls (—SH) may promote shuffling.

The formation of a thioether and racemization of a disulphide bond can occur under basic conditions, in production or storage, through a beta elimination of di-sulphide bridges back to cysteine residues via a dehydroalanine and persulfide intermediate. Subsequent crosslinking of dehydroalanine and cysteine may result in the formation of a thioether bond or the free cysteine residues may reform a disulphide bond with a mixture of D- and L-cysteine.

Trisulfides may result from insertion of a sulfur atom into a disulphide bond (Cys-S—S—S-Cys) and may be formed due to the presence of hydrogen sulphide in production cell culture.

N-terminal glutamine (Q) and glutamate (glutamic acid) (E) in the heavy chain and/or light chain may form pyroglutamate (pGlu) via cyclization. pGlu formation may form in the production bioreactor, but it can also be formed, for example, non-enzymatically, depending on pH and temperature of processing and storage conditions. Cyclization of N-terminal Q or E is commonly observed in natural human antibodies.

C-terminal lysine cleavage is an enzymatic reaction catalyzed by carboxypeptidases, and is commonly observed in recombinant and natural human antibodies. Variants of this process include removal of lysine from one or both heavy chains due to cellular enzymes from the recombinant host cell. Administration to the human subject/patient is likely to result in the removal of any remaining C-terminal lysine.

The present disclosure encompasses antibodies which may have been subjected to, or have undergone, one or more of a post-translational modification described herein. Exemplary compositions may comprise a mixture or blend of antibodies: 1) with and without post-translational modifications (1 or more), or 2) with more than one type of a post-translational modifications described herein.

The composition may comprise a mixture of antibody variants and post-translational modification variants. For example, the antibody composition may comprise one or more, such as two or more of oxidation variants, deamidation variants, isomerized variants, N-terminal pyro-glutamate variants, and C-terminal lysine cleaved variants.

For example, in one embodiment, a composition may comprise a mixture of antibodies, wherein 10% of the antibody in the mixture comprises the amino acid sequence of SEQ ID NO: 9 and 10, and 90% of the antibody in the mixture comprises the amino acid sequence of SEQ ID NO: 9 and 10 with a C-terminal lysine cleavage.

In another exemplary embodiment, a composition may comprise a mixture of antibodies, wherein 10% of the antibody in the mixture comprises the amino acid sequence of SEQ ID NO: 9 and 10, 90% of the antibody in the mixture comprises the amino acid sequence of SEQ ID NO: 9 and 10 with a C-terminal lysine cleavage, and of that 100% total antibody mixture, up to 100% of the N-terminal glutamine is cyclized to pyro-glutamate.

In another exemplary embodiment, a composition may comprise a mixture of antibodies, wherein 10% of the antibody in the mixture comprises the amino acid sequence of SEQ ID NO: 9 and 10, 90% of the antibody in the mixture comprises the amino acid sequence of SEQ ID NO: 9 and 10 with a C-terminal lysine cleavage, and of that 100% total antibody mixture, up to 100% is N-terminal pyro-glutamate and up to 23% is isomerized at D103 at CDRH3.

In yet another exemplary embodiment, a composition comprises a mixture of antibodies, wherein 20% of the antibody in the mixture comprises the amino acid sequence of SEQ ID NO: 9 and 10, 80% of the antibody in the mixture comprises the amino acid sequence of SEQ ID NO: 9 and 10 with variant N103 at CDRH3, and of that 100% total antibody mixture, up to 37% of the antibody is oxidized at amino acid M34 CDRH1.

In one embodiment, a post-translational modification described herein, does not result in a significant change in antigen binding affinity, biological activity, pharmacokinetics (PK)/pharmacodynamics (PD), aggregation, immunogenicity, and/or binding to an Fc receptor, except where specified and described as a product-related impurity.

“Function” or “activity” as described herein is defined as one or more of 1) binding to BCMA, 2) binding to FcγRIIIa, and/or 3) binding to FcRn. In one embodiment, “reduced function” or “reduced activity” means that binding to BCMA, binding to FcγRIIIa, or binding to FcRn is reduced as a percentage compared to a reference standard, and is significant over assay variability. For example, reduced function or activity can be described as a reduction of ≥5%, ≥10%, ≥15%, ≥20%, ≥25%, ≥30%, ≥35%, ≥40%, ≥45%, or ≥50%.

In one embodiment, an anti-BCMA antibody comprises an antibody that is at least about 90% identical to the amino acid sequences of SEQ ID NO:9 and SEQ ID NO:10 and includes all post-translational modifications, if any, of the antibody.

In another embodiment, an anti-BCMA antibody comprises belantamab and all post-translational modifications if any.

Antibody variants are commonly observed when the composition of antibodies is analyzed by charged based-separation techniques such as isoelectric focusing (IEF) gel electrophoresis, capillary isoelectric focusing (cIEF) gel electrophoresis, cation exchange chromatography (CEX) and anion exchange chromatography (AEX).

### Pharmaceutical Compositions

A composition described herein can be in the form of a pharmaceutical composition. A “pharmaceutical composition” may comprise a composition described herein (e.g., active ingredient), and one or more pharmaceutically acceptable excipients. The excipient(s) must be acceptable in the sense of being compatible with the other ingredients of the formulation, capable of pharmaceutical formulation, not deleterious to the recipient thereof, and/or do not interfere with the efficacy of the active ingredient.

As used herein, “pharmaceutically acceptable excipient” may include any and all solvents, diluents, carriers, dispersion media, coatings, antibacterial and antifungal agents, isotonic and/or absorption delaying agents. Examples of pharmaceutically acceptable excipients include one or more of buffering agents, water, saline, phosphate buffered saline, dextrose, glycerol, ethanol and the like, as well as combinations thereof. In many cases, it will be preferable to include isotonic agents, for example, polyol, sugars, polyalcohols such as mannitol, sorbitol, or sodium chloride in the composition. preservatives; co-solvents; antioxidants including ascorbic acid and methionine; chelating agents such as EDTA; metal complexes (e.g., Zn2+-protein complexes); biodegradable polymers; and/or salt-forming counterions such as sodium or potassium.

The precise nature of the excipient or other material may depend on the route of administration, which may be, for example, oral, rectal, nasal, topical (including buccal and sublingual), vaginal, parenteral (including subcutaneous, intramuscular, intravenous, intradermal, intrathecal, and epidural), and intratumorally. It will be appreciated that the preferred excipient may vary with, for example, the condition of the recipient and the disease to be treated.

A mixture of excipients and concentrations of each together form a “pharmaceutical formulation” (or “formulation”). The formulation may be in liquid form or lyophilized form. A composition in a liquid formulation may be filled into containers and frozen. In certain embodiments, aliquots of the frozen formulation comprising the composition may be lyophilized. Lyophilizate may be reconstituted by the addition of water or other aqueous solution to produce a reconstituted formulation comprising the composition.

In some embodiments, an anti-BMCA antigen binding protein is present in a formulation at a concentration of at least about 10 mg/mL or at least about 20 mg/mL. In some embodiments, an anti-BMCA antigen binding protein is present in a formulation at a concentration of between about 20 mg/mL to about 100 mg/mL, or about 20 mg/mL to about 60 mg/mL. In certain embodiments, the concentration of an anti-BCMA antigen binding protein in the formulation is about 20 mg/mL, about 25 mg/mL, about 50 mg/mL, about 60 mg/mL, or about 100 mg/mL. In one embodiment, an anti-BMCA antigen binding protein is present in a liquid formulation at a concentration of about 20 mg/mL or about 25 mg/mL. In another embodiment, an anti-BMCA antigen binding protein is present in a lyophilized formulation at a concentration of about 50 mg/mL or about 60 mg/mL. In yet another embodiment, the anti-BMCA antigen binding protein is present in a reconstituted formulation at a concentration of about 50 mg/mL.

In certain embodiments, a buffering agent is a citrate buffer. Citrate buffer can be achieved, for example, by the use of a conjugate acid/conjugate base system (sodium citrate/citric acid) or by HCl titration of a sodium citrate solution. In certain embodiments, the concentration of a citrate buffer is about 10 mM to about 30 mM. In preferred embodiments, the concentration of a citrate buffer is 25 mM. In some embodiments, a buffering agent is a histidine buffer at a concentration from about 5 mM to about 35 mM.

A buffering agent may be used to help maintain preferred pH ranges. In certain embodiments, the pH of a formulation is about 5.5 to about 7 or about 5.9 to about 6.5, preferably pH 6.2.

In some embodiments, a formulation comprises a polyol. In some embodiments, a polyol is a sugar, and preferably a non-reducing sugar. In some embodiments, a non-reducing sugar is trehalose. In some embodiments, the formulation comprises trehalose in the range from about from about 120 mM to about 240 mM. In yet another embodiment, the formulation comprises trehalose at about 200 mM.

In one embodiment, a formulation comprises a chelating agent. In another embodiment, a chelating agent is EDTA. In certain embodiments, the formulation comprises EDTA at a concentration of 0.01 mM to about 0.1 mM. In yet another embodiment, the formulation comprises EDTA at a concentration of 0.05 mM.

In some embodiments, a formulation comprises a surfactant. “Surfactants” are surface active agents that can exert their effect at surfaces of solid-solid, solid-liquid, liquid-liquid, and liquid-air interfaces because of their chemical composition, containing both hydrophilic and hydrophobic groups. Surfactants may reduce the concentration of proteins in dilute solutions at the air-water and/or water-solid interfaces where proteins can be adsorbed and potentially aggregated. Surfactants can bind to hydrophobic interfaces in protein formulations. Some parentally acceptable nonionic surfactants comprise either polysorbate or polyether groups. Polysorbate 20 and 80 are suitable surfactant stabilizers in formulations of the disclosure. In some embodiments, a formulation comprises polysorbate 20 or polysorbate 80 at about 0.01% to about 0.05%. In yet another embodiment, a formulation comprises polysorbate 20 or polysorbate 80 at about 0.02%. In a preferred embodiment, a formulation comprises polysorbate 80 at about 0.02%.

One aspect of the disclosure is drawn to a formulation that comprises from about 20 mg/mL to about 100 mg/mL of the anti-BCMA ADC, from about 10 mM to about 25 mM of a buffering agent, from about 120 mM to about 240 mM of a polyol, and a pH in the range of 5.5 to 6.5.

In one embodiment, a formulation comprises an anti-BCMA ADC at about 20 mg/mL to about 60 mg/mL, citrate buffer at about 10 mM to about 30 mM, trehalose at about 120 mM to about 240 mM, EDTA at about 0.01 mM to about 0.1 mM, polysorbate 20 or polysorbate 80 at about 0.01% to about 0.05%, at a pH of about 5.9 to about 6.5.

In one embodiment, a composition comprises an ADC in a formulation, wherein the antibody comprises a CDRH1 with the amino acid sequence set forth in SEQ ID NO:1, a CDRH2 with the amino acid sequence set forth in SEQ ID NO:2, a CDRH3 with the amino acid sequence set forth in SEQ ID NO:3, a CDRL1 with the amino acid sequence set forth in SEQ ID NO:4, a CDRL2 with the amino acid sequence set forth in SEQ ID NO:5, and a CDRL3 with the amino acid sequence set forth in SEQ ID NO:6; wherein the cytotoxin is MMAE or MMAF; and wherein the formulation comprises the ADC at about 20 mg/mL to about 60 mg/mL, citrate buffer at about 10 mM to about 30 mM, trehalose at about 120 mM to about 240 mM, EDTA at about 0.01 mM to about 0.1 mM, polysorbate 20 or polysorbate 80 at about 0.01% to about 0.05%, at a pH of about 5.9 to about 6.5.

In one embodiment, a composition comprises an ADC in a formulation, wherein the antibody comprises a V_{H} with the amino acid sequence set forth in SEQ ID NO:7, and a V_{L} with the amino acid sequence set forth in SEQ ID NO:8; wherein the cytotoxin is MMAF or MMAE; and wherein the formulation comprises the ADC at about 20 mg/mL to about 60 mg/mL, citrate buffer at about 10 mM to about 30 mM, trehalose at about 120 mM to about 240 mM, EDTA at about 0.01 mM to about 0.1 mM, polysorbate 20 or polysorbate 80 at about 0.01% to about 0.05%, at a pH of about 5.9 to about 6.5.

In one embodiment, a composition comprises an ADC in a formulation, wherein the ADC is belantamab mafodotin; and wherein the formulation comprises belantamab mafodotin at about 20 mg/mL to about 60 mg/mL, citrate buffer at about 10 mM to about 30 mM, trehalose at about 120 mM to about 240 mM, EDTA at about 0.01 mM to about 0.1 mM, polysorbate 20 or polysorbate 80 at about 0.01% to about 0.05%, at a pH of about 5.9 to about 6.5.

In one embodiment, a composition comprises belantamab mafodotin in a formulation comprising about 20 mg/mL, about 25 mg/mL, about 50 mg/mL, or 60 mg/mL belantamab mafodotin, 25 mM citrate buffer, 200 mM trehalose, 0.05 mM disodium EDTA, 0.02% polysorbate or 80 polysorbate 80 at a pH of about 5.9 to about 6.5.

In some embodiments, each mL of a composition disclosed herein comprises belantamab mafodotin (50 mg), citric acid (0.42 mg), disodium edetate dihydrate (0.019 mg), polysorbate 80 (0.2 mg), trehalose dihydrate (75.6 mg), and trisodium citrate dihydrate (6.7 mg) at a pH of about 6.2.

A “stable” formulation is one in which the protein therein essentially retains its physical and/or chemical stability during manufacturing, transport, storage, and administration. Stability can be measured at a selected temperature for a selected time period. For example, for a product stored at a recommended temperature of 2° C. to 8° C., the formulation is stable at room temperature, about 30° C., or at 40° C., for at least 1 month and/or stable at about 2 to 8° C. for at least 1 year and preferably for at least 2 years. For example, the extent of aggregation during storage can be used as an indicator of protein stability. Thus, a “stable” formulation may be one wherein, for example, less than about 10% and preferably less than about 5% of the protein is present as an aggregate in the formulation. Various analytical techniques for measuring protein stability are available in the art and are reviewed, for example, in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993).

In certain aspects of the disclosure, a formulation allows the composition to remain stable to freezing, thawing, and/or mixing.

In yet another aspect, the present disclosure is directed to an article of manufacture, e.g., a kit, comprising a container holding a composition in a formulation described herein. In one aspect there is provided an injection device comprising the formulation. The injection device may comprise a pen injector device or an autoinjector device. In one embodiment, the formulation is contained in a prefilled syringe.

### Methods of Treatment and Compositions for Use

Compositions of the present disclosure may provide a therapeutic approach to the treatment of B-cell related disorders or diseases, such as antibody-mediated or plasma cell mediated diseases, or plasma cell malignancies (e.g., cancers such as multiple myeloma), or other disease that may be treated by an anti-BCMA ADC. In particular it is an object of the present disclosure to provide compositions comprising an anti-BCMA ADC that specifically bind to BCMA (e.g., human BCMA) and modulate (e.g. inhibit or block) the interaction between BCMA and its ligands such as BAFF and/or APRIL in the treatment of diseases and disorders responsive to modulation of that interaction.

In another aspect of the present disclosure, there is provided a method of treating a subject (e.g. human patient) afflicted with a B-cell related disorders or diseases, such as antibody-mediated or plasma cell mediated diseases, or plasma cell malignancies (e.g. cancers such as multiple myeloma), such method comprises the step of administering to said subject a therapeutically effective amount of an anti-BCMA ADC composition as described herein.

In yet another embodiment, the present disclosure provides a method of treating a cancer patient, which method comprises the step of administering to said patient a therapeutically effective amount of an anti-BCMA ADC composition described herein.

As used herein, the terms “cancer,” and “tumor” are used interchangeably and, in either the singular or plural form, refer to cells that have undergone a transformation, such as malignant transformation, that makes them pathological to the host organism. Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient. Tumors may be a hematopoietic (or hematologic or hematological or blood-related) cancer, for example, cancers derived from blood cells or immune cells, which may be referred to as “liquid tumors.” Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like.

The cancer may be any in which an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia (AML). Myeloid malignancies also include myeloproliferative disorders (MPD) which include, but are not limited to, chronic myelogenous (or myeloid) leukemia (CML), chronic myelomonocytic leukemia (CMML), essential thrombocythemia (or thrombocytosis), and polcythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (MFS) with or without agnogenic myeloid metaplasia, among others.

Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate-grade (or aggressive) or high-grade (very aggressive). Indolent B-cell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphoma s(T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome, among others.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenstroem's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

In one embodiment, the cancer is selected from the group consisting of colorectal cancer (CRC), gastric, esophageal, cervical, bladder, breast, head and neck, ovarian, melanoma, renal cell carcinoma (RCC), EC squamous cell, non-small cell lung carcinoma, mesothelioma, pancreatic, and prostate cancer.

The term “treating” and derivatives thereof as used herein, is meant to include therapeutic therapy. In reference to a particular condition, treating means: (1) to ameliorate the condition or one or more of the biological manifestations of the condition; (2) to interfere with (a) one or more points in the biological cascade that leads to or is responsible for the condition or (b) one or more of the biological manifestations of the condition; (3) to alleviate one or more of the symptoms, effects or side effects associated with the condition or one or more of the symptoms, effects or side effects associated with the condition or treatment thereof; (4) to slow the progression of the condition or one or more of the biological manifestations of the condition and/or (5) to cure said condition or one or more of the biological manifestations of the condition by eliminating or reducing to undetectable levels one or more of the biological manifestations of the condition for a period of time considered to be a state of remission for that manifestation without additional treatment over the period of remission. One skilled in the art will understand the duration of time considered to be remission for a particular disease or condition.

B-cell disorders can be divided into defects of B-cell development/immunoglobulin production (e.g., immunodeficiencies) and excessive/uncontrolled proliferation (e.g. lymphomas, leukemias). As used herein, B-cell disorder refers to both types of diseases, and methods are provided for treating B-cell disorders with the compositions described herein.

In a particular aspect, the disease or disorder is Multiple Myeloma (MM), Chronic Lymphocytic Leukaemia (CLL), Solitary Plasmacytoma (Bone, Extramedullary), amyloidosis (AL), Smoldering Multiple Myeloma (SMM), Solitary Plasmacytoma (Bone, Extramedullary), or Waldenstrom's Macroglobulinemia.

Prophylactic therapy is also contemplated. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

“Subject” or “patient” are used interchangeably herein and are defined broadly to include any person in need of treatment, for example, a person in need of cancer treatment. A subject may include a mammal. In one embodiment, the subject is a human patient. The subject in need of cancer treatment may include patients from a variety of stages including newly diagnosed, relapsed, refractory, progressive disease, remission, and others. The subject in need of cancer treatment may also include patients who have undergone stem cell transplant or who are considered transplant ineligible.

Subjects may be pre-screened in order to be selected for treatment with the compositions described herein. In one embodiment, a sample from the subject is tested for expression of BCMA prior to treatment with the compositions described herein.

Subjects may have had at least one prior cancer therapy before being treated with the compositions of the present disclosure. In one embodiment, the subject has been treated with at least 1, at least 2, at least 3, at least 4, at least 5, at least 6, or at least 7 prior cancer therapies before being treated with the compositions of the present disclosure.

In another embodiment, the subject has newly diagnosed cancer and has had 0 prior therapies before being treated with the compositions of the present disclosure.

The compositions of the disclosure may be administered by any appropriate route. For some compositions, suitable routes include oral, rectal, nasal, topical (including buccal and sublingual), vaginal, parenteral (including subcutaneous, intramuscular, intravenous, intradermal, intrathecal, and epidural), and intratumorally. It will be appreciated that the preferred route may vary with, for example, the condition of the recipient and the cancer to be treated.

In certain embodiments, a composition of the disclosure are administered as a pharmaceutical composition.

The term “administering” as used herein is meant to refer to the delivery of the compositions described herein to achieve a therapeutic objective. The compositions may be administered at an administration interval for a period sufficient to achieve clinical benefit. The composition may be administered to the subject in such a way as to target therapy to a particular site.

In some embodiments, the composition is administered by injection. Therefore, in one aspect there is provided an injection device comprising the composition, pharmaceutical composition or formulation of the disclosure. The injection device may comprise a pen injector device or an autoinjector device.

The term “therapeutically effective amount” or “therapeutically effective dose” of a composition as used herein refers to an amount effective in the prevention or treatment or alleviation of a symptom of a B-cell mediated disorder or disorder. Therapeutically effective amounts and treatment regimens are generally determined empirically and may be dependent on factors, such as the age, weight, and health status of the patient and disease or disorder to be treated. Such factors are within the purview of the attending physician.

The appropriate therapeutically effective dose of the composition comprising an anti-BCMA ADC will be determined readily by those of skill in the art. Suitable doses of the compositions described herein may be calculated for patients according to their weight, for example suitable doses may be in the range of about 0.1 mg/kg to about 20 mg/kg, for example about 1 mg/kg to about 20 mg/kg, for example about 10 mg/kg to about 20 mg/kg or for example about 1 mg/kg to about 15 mg/kg, for example about 10 mg/kg to about 15 mg/kg.

In one embodiment, a therapeutically effective dose of the composition comprising an anti-BCMA ADC is in the range of about 0.03 mg/kg to about 4.6 mg/kg. In yet another embodiment, a therapeutically effective dose of the composition comprising an anti-BCMA ADC is at least about 0.03 mg/kg, 0.06 mg/kg, 0.12 mg/kg, 0.24 mg/kg, 0.48 mg/kg, 0.96 mg/kg, 1 mg/kg, 1.92 mg/kg, 3.4 mg/kg, or 4.6 mg/kg. In yet another embodiment, a therapeutically effective dose of the composition comprising an anti-BCMA ADC is 1.9 mg/kg, 2.5 mg/kg or 3.4 mg/kg.

In certain embodiments, a composition can be co-administered to a subject with

one or more additional therapeutic agents. In another embodiment, a composition can be co-administered to a subject with one or more additional cancer therapeutics. The additional cancer therapeutic agent may include, but is not limited to, other immunomodulatory drugs, therapeutic antibodies (e.g., an anti-CD38 antibody such as daratumumab), CAR-T therapeutics, BiTEs, HDAC inhibitors, proteasome inhibitors (e.g., bortezomib), anti-inflammatory compounds, and immunomodulatory imide drugs (IMiD) (e.g., thalidomide and analogs thereof).

“Co-administered” means the administration of two or more different pharmaceutical compositions or treatments (e.g., radiation treatment) that are administered to a subject by combination in the same pharmaceutical composition or separate pharmaceutical compositions. Thus co-administration involves administration at the same time of a single pharmaceutical composition comprising two or more pharmaceutical agents or administration of two or more different compositions to the same subject at the same or different times.

In one aspect of the disclosure, the disclosure provides a method of treating a B-cell disease or disorder in a subject in need thereof by administering a therapeutically effective dose of any of the compositions comprising an anti-BCMA ADC as described herein.

In one aspect of the disclosure, the disclosure provides a composition as described herein for use in the treatment of B-cell diseases or disorders. In another aspect of the disclosure, the disclosure provides a composition as described herein for use in the treatment of cancer.

In one aspect of the disclosure, provided is the use of a composition in the manufacture of a medicament for use in the treatment of B-cell diseases or disorders. In another aspect of the disclosure, provided is the use of a composition in the manufacture of a medicament for use in the treatment of cancer.

All patent and literature references disclosed herein are expressly and entirely incorporated herein by reference.

### EXAMPLES

### Reagents and Equipment

Belantamab was produced and purified using a standard mAb manufacturing procedure. Belantamab was then conjugated with MMAF to produce belantamab mafodotin ADC drug substance. All samples were stored in formulation buffer at −80° C. prior to analysis.

Maleimidocaproyl monomethylauristatin F (mcMMAF) was purchased from MedChemExpress (Monmouth Junction, NJ). Isotopically-labeled water (H₂¹⁸O, 97%), dithiothreitol (DTT), sodium iodoacetate (IAA), tris(2-carboxyethyl)phosphine (TCEP), calcium chloride, and dimethyl sulfoxide (DMSO) were purchased from Sigma-Aldrich (St. Louis, MO). Guanidine HCl was purchased from MP Biomedicals (Irvine, CA). Size exclusion chromatography (SEC) spin columns were purchased from Bio-Rad (Hercules, CA), and trypsin was purchased from Worthington Biochemical (Lakewood, NJ). Tris base, ethylenediaminetetraacetic acid (EDTA) disodium salt, hydro-chloric acid, and LC-MS-grade trifluoroacetic acid (TFA), water (H₂O), and acetonitrile (ACN) were purchased from ThermoFisher Scientific (Waltham, MA).

Liquid chromatography tandem mass spectrometry (LC-MS/MS) analysis was performed on a Waters Acquity UPLC system equipped with a Waters BEH 300 C18 column (2.1×150 mm, 1.7 μm particle) connected to a Thermo Scientific Orbitrap XL mass spectrometer.

Reduced LC-MS analysis was performed on a Waters Acquity UPLC system equipped with a Waters BEH200 SEC (4.6×150 mm, 1.7 μm particle) connected to a either a Xevo G2-XS mass spectrometer.

### Example 1: Stable Isotope Labeling of MMAF

### Sample Preparation

MMAF (20 mg) was dissolved in 120 μL of acetonitrile and mixed with 80 μL of 2.5% (v/v) TFA in H₂¹⁸O to give final reaction conditions of 100 mg/mL MMAF in 1% TFA 60:40 ACN:H₂¹⁸O. The solution was reacted at room temperature protected from light for 14 days. Aliquots (10 μL) were frozen at −80° C. until analysis.

The isotopic purity of labeled MMAF was determined by diluting aliquots 1000× (0.1 mg/mL) with 50:50 ACN:H₂O and analyzing using reversed-phase liquid chromatographic separation using a linear gradient from 0% to 100% B over five minutes at a flow rate of 0.2 mL/min. Mobile phase A was 0.1% TFA in water, and mobile phase B was 0.09% TFA in ACN. The column was flushed with 100% B for three minutes and re-equilibrated with 0% B for twelve minutes after each injection. The column temperature was 30° C., the autosampler temperature was 5° C., and the injection volume was 5 μL.

MS analysis was performed post-UV detection (215 nm) operating in data-dependent acquisition mode (1 MS scan followed by 2 MS/MS scans). Electrospray ionization (ESI) spray voltage was 4.5 kV, sheath gas flow rate was 40 L/min, auxiliary gas flow rate was 5 L/min, capillary voltage was 60 V, capillary temperature was 250° C., tube lens was 120 V, and scan range was m/z 250 to 2000. Data were examined using Thermo Xcalibur software.

### Results and Discussion

Stable isotope labeling of MMAF was performed using an acid-mediated solvent exchange mechanism previously described (Liu et al., Advances and applications of stable isotope labeling-based methods for proteome relative quantitation. Trends in Anal. Chem. 2020, 124, 115815). Briefly, two 18-oxygen atoms can be exchanged from isotopically-labeled water into a carboxylic acid functional group through an ortho-acid intermediate when reacted under acidic conditions. Several labeling optimization conditions were investigated including acid concentration [0.1%-10% (v/v)], acid type (TFA or FA), reaction temperature (room temperature, 37° C., 70° C.), and organic phase (ACN or DMSO). Reaction times ranged from 1 hour-6 weeks with higher acid concentrations and temperatures resulting in faster labeling kinetics as expected. Optimization experiments were performed using 0.1 mg/mL MMAF in 50:50 ACN:H₂¹⁸O. Concentrated batches (100 mg/mL) were labeled in 60:40 ACN:H₂¹⁸O due to a thin organic solvent layer forming in 50:50 ACN:H₂¹⁸O.

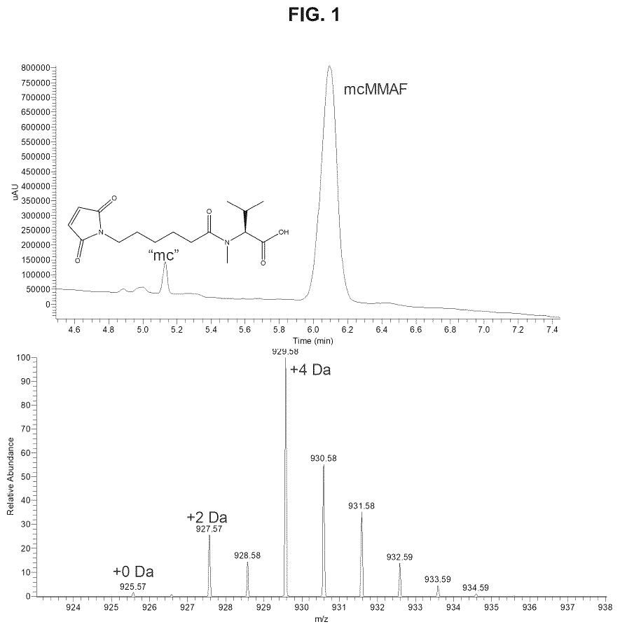

Minimization of MMAF hydrolysis (FIG. 1) was the primary factor in optimizing labeling reaction conditions due to the maleimide-containing fragment potentially competing with labeled MMAF for unoccupied ADC conjugation sites. Labeling reactions were not able to proceed to equilibrium with this limitation and were stopped when minimal unlabeled (+0 Da) MMAF remained. This resulted in a mixture of singly-labeled (+2 Da) and doubly-labeled (+4 Da) MMAF. This discrepancy was accounted for during MS data processing. Final labeling reaction conditions (1% TFA at room temperature for 14 days) were ultimately chosen based on sufficient labeling (minimization of +0 Da MMAF) and minimal hydrolysis (<5%).

### Example 2: ADC Reduction and Conjugation with Isotopically-Labeled MMAF

### Sample Preparation

Belantamab mafodotin (250 μg, 10 mg/mL) was partially reduced with 1.25 μL of 1 M DTT at 37° C. for 15 minutes in formulation buffer (pH 6.2). Excess DTT was removed by eluting samples through SEC spin columns equilibrated with formulation buffer. Samples were then conjugated with 1 μL of 100 mg/mL isotopically-labeled MMAF at room temperature for 30 minutes. Excess MMAF was removed by eluting samples through SEC spin columns equilibrated with formulation buffer.

Conjugation efficiency was determined by diluting samples to 1 mg/mL with water and analyzing using an isocratic size exclusion liquid chromatographic separation using a 0.1% TFA in 65:35 ACN:H₂O mobile phase at a flow rate of 0.2 mL/min. The column temperature was 25° C., the autosampler temperature was 5° C., and the injection volume was 1 μL.

MS analysis was performed in sensitivity mode post-UV detection (215 nm). Electrospray ionization (ESI) spray voltage was 2.2 kV, sampling cone was 120, source temperature was 150° C., source offset was 80 V, desolvation temperature was 500° C., cone gas flow was 60 L/hr, and desolvation gas flow was 800 L/hr. Scan range was m/z 700 to 5000, and scan time was 1 s. Data were examined using Waters MassLynx software.

### Results and Discussion

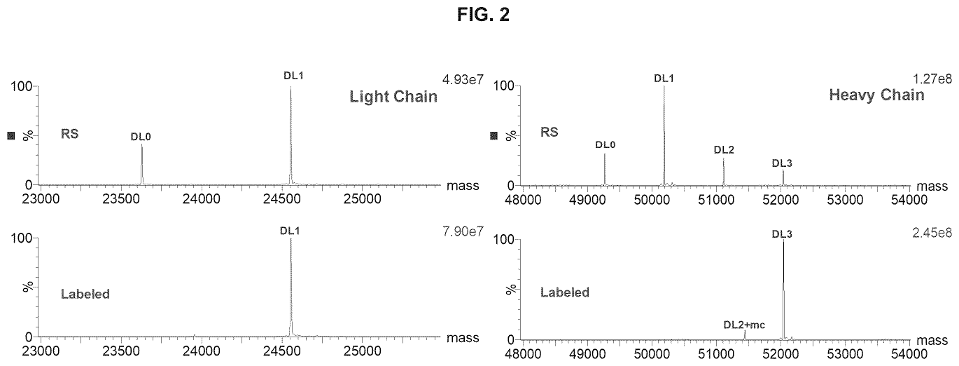

Reduction/conjugation schemes: briefly, belantamab is partially reduced with TCEP and subsequently conjugated with MMAF. TCEP is typically used in favor of DTT to avoid competing side-reactions between the maleimide and thiol groups of MMAF and DTT, respectively. Initial reduction/conjugation experiments did confirm TCEP allowed for greater conjugation efficiency compared to DTT; however, subsequent peptide mapping analysis using a legacy method called for DTT to be used for complete reduction of all remaining disulfide bonds. DTT was chosen for both reduction steps, and excess DTT was removed with an SEC spin column before subsequent MMAF conjugation resulting in completely conjugated light and heavy chains with minimal hydrolysis fragment conjugation (FIG. 2).

### Example 3: Stable Isotope Labeling Peptide Mapping LC-MS/MS Analysis of Belantamab Mafodotin

### Sample Preparation

Belantamab mafodotin (250 μg, 10 mg/mL) was reduced and conjugated with isotopically-labeled MMAF as described in Example 2. Samples were then prepped with a standard peptide mapping procedure by evaporating to near dryness (˜5 μL) and denaturing by adding 60 μL of denaturation buffer (6 M guanidine HCl, 1.2 M Tris HCl, 2.5 mM Na₂EDTA, pH 7.5) and vortexing at room temperature for 2 minutes. Samples were reduced by adding 3 μL of 1 M DTT and incubating at room temperature for 20 minutes and then alkylated by adding 7.2 μL of 1 M IAA and incubating at room temperature for 30 minutes. Alkylation was quenched by adding 4.2 μL of 1 M DTT. Samples were then buffer exchanged using SEC spin columns into digestion buffer (50 mM Tris, 1 mM CaCl₂, pH 7.5) and digested by adding 2.5 μL of 5 mg/mL trypsin (1:20 enzyme:protein) and incubating at 37° C. for 30 min. Digestion was quenched by adding 3 μL of 1 N HCl, and 10 μL was injected for LC-MS/MS analysis.

Samples were analyzed using identical LC-MS/MS conditions as described in Example 2 except for a two-step gradient from 0% to 40% B in 90 minutes followed by 40% to 60% B in 10 min. The column was flushed with 100% B for 12 minutes and re-equilibrated with 0% B for 18 minutes after each injection. Sequence coverage and PTM data were analyzed using Protein Metrics Byos software (Cupertino, CA). Isotopic conjugation data were analyzed using Skyline software (University of Washington).

### Results and Discussion

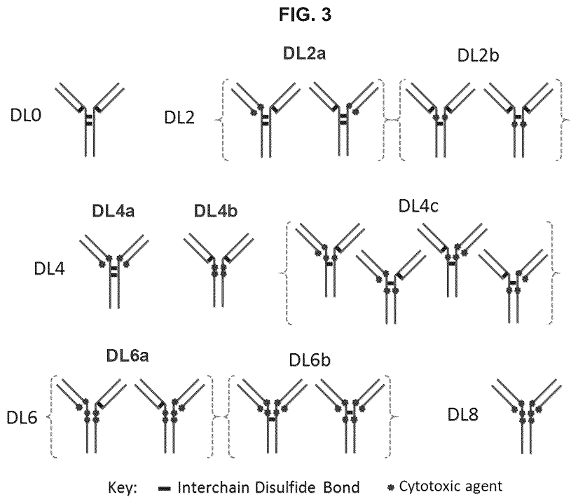

Cysteine-conjugated ADCs are typically conjugated at the cysteine residues involved in interchain disulfide bonds. Reduction of these disulfide bonds results in eight potential conjugation sites (one on each light chain, one in each heavy chain Fab region, and two in each heavy chain hinge region). Even-numbered drug-loaded species are typically seen due to each disulfide bond reduction resulting in two free sulfhydryl groups. This process results in drug-loaded species ranging from DL0 to DL8 along with possible positional isomers (FIG. 3).

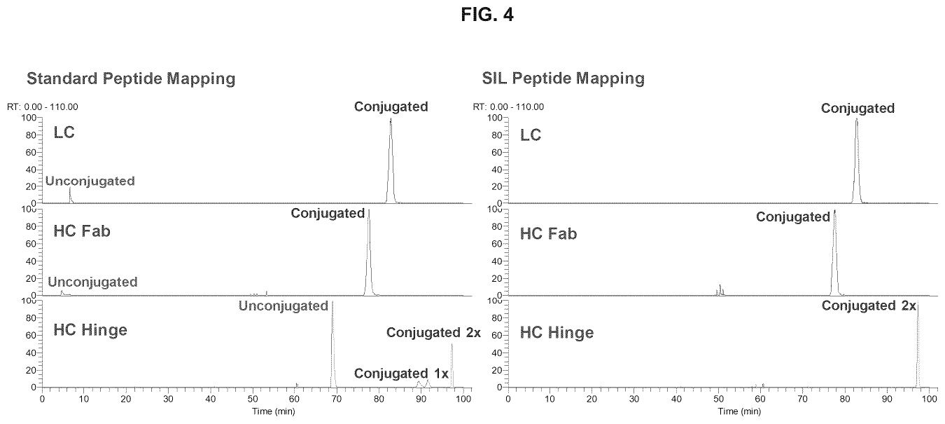

SIL peptide mapping produced liquid chromatography peaks that contained both natural and labeled versions of conjugated peptides compared to standard peptide mapping which produced separate peaks for native and conjugated peptides (FIG. 4). Conjugated peptide peak data for SIL samples were processed using Skyline software by integrating extracted ion chromatograms (XICs) to produce peak areas for individual isotopes of interest.

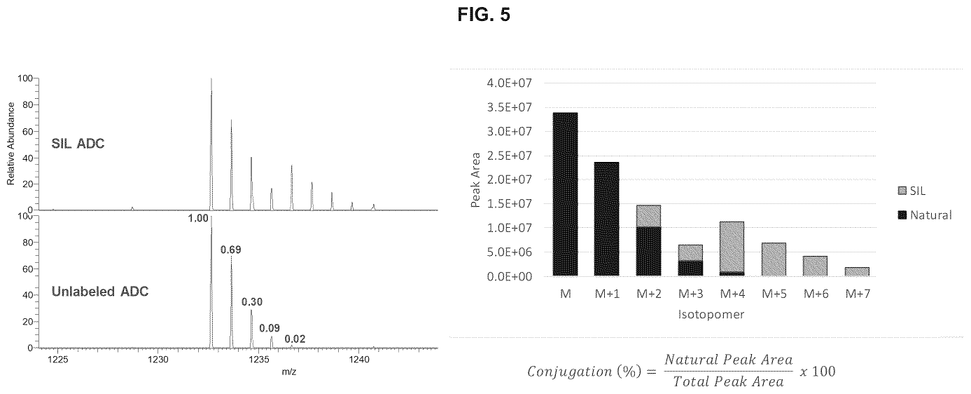

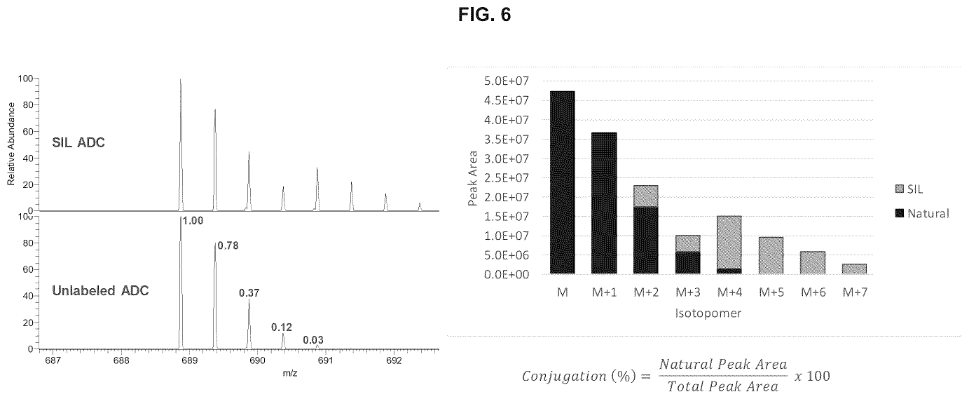

SIL did not induce a large enough mass change to completely separate the isotopic envelopes of the natural and SIL peptides. This resulted in mixtures of isotope isomers (isotopomers) due to the overlap of isobaric isotopes corresponding to natural or SIL conjugated peptides. Therefore, theoretical isotope ratios calculated from an unlabeled ADC sample were used to calculate the natural isotope contribution of the M+2-M+4 isotopomers for the light chain and heavy chain Fab peptides (FIG. 5 and FIG. 6). This calculation also corrected for sites that were conjugated with singly-labeled (+2 Da) MMAF. The summed natural isotopomer peak areas were then divided by the total isotopomer peak area to determine the peptide conjugation level.

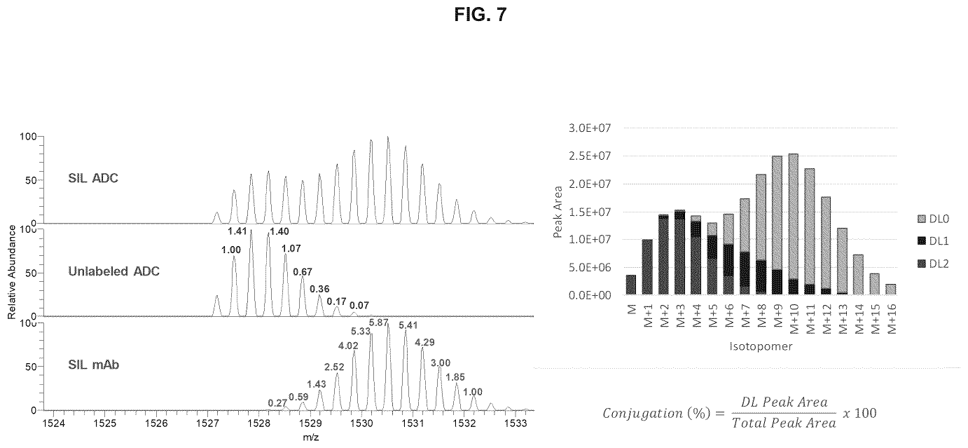

Theoretical isotope ratios for the doubly-conjugated (DL2, C230 and C233) and native (DL0) versions of the hinge peptide were calculated from an unlabeled ADC sample and a SIL mAb intermediate sample, respectively (FIG. 7). The DL0 isotope ratios for the mAb sample accounted for isotopomer contributions from DL0 peptides labeled with all combinations of SIL MMAF at the two available hinge conjugation sites. These DL2 and DL0 theoretical isotope ratios were used to calculate the isotopomer contributions of each peptide form. First, the M and M+1 isotopomer peak areas were assumed to be completely associated with DL2 peptides. The M+1 peak area was then multiplied by the DL2 relative theoretical isotope ratio of each isotopomer to yield the DL2 contribution to the M+2-M+14 isotopomers. Next, the M+15 and M+16 isotopomers were assumed to be completely associated with DL0 peptides. The M+15 peak area was then multiplied by the DL0 relative theoretical isotope ratio of each isotopomer to yield the DL0 contribution to the M+2-M+14 isotopomers. Finally, both the DL2 and DL0 isotopomer peak areas were subtracted from each total isotopomer peak area to yield the singly-conjugated (DL1, C230 or C233) hinge peptide contributions. The summed isotopomer peak areas of each form were then divided by the total isotopomer peak area to determine the level of each hinge-conjugation peptide form. Interestingly, this data processing method was compared to a more rigorous algorithm described by Jennings and Matthews (Determination of Complex Isotopomer Patterns in Isotopically Labeled Compounds by Mass Spectrometry. Anal. Chem. 2005, 77, 6435-6444) with both methods yielding similar results.

Comparison of SIL Peptide Mapping with Standard Peptide Mapping

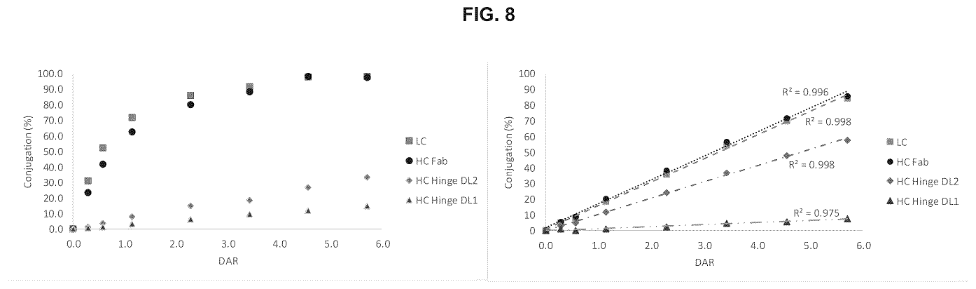

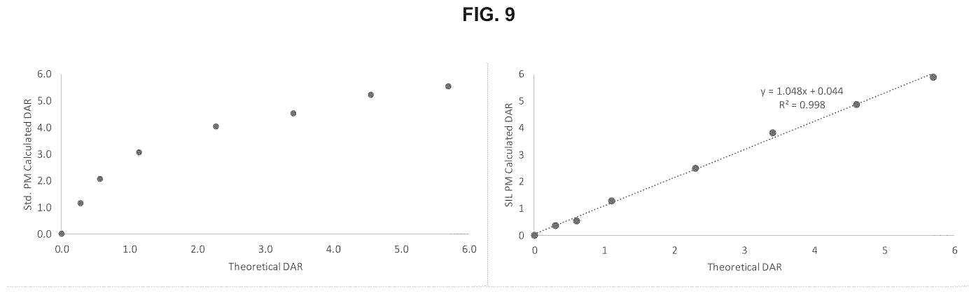

Method linearity was investigated by testing samples comprised of linear combinations of belantamab (mAb) and belantamab mafodotin (ADC) to produce samples with DARs ranging from 0.0-5.7 (Table 1 and Table 2). Standard peptide mapping suffered from decreased sensitivity and linearity when quantifying light chain and heavy chain Fab conjugation levels, showing the impracticality of using relative quantitation between native and conjugated peptides. Meanwhile, SIL peptide mapping resulted in excellent linearity (R²≥0.996) for both sites across all samples (FIG. 8). Both methods gave linear results for singly- and doubly-conjugated hinge sites; however, SIL peptide mapping showed a greater discrepancy between the amount of doubly-conjugated hinge compared to singly-conjugated hinge. Singly-conjugated hinge was not detected as a major isoform for any drug-loaded version of belantamab mafodotin by orthogonal methods. This result correlated well with the SIL peptide mapping analysis of drug-loaded fractions described in Experiment 4. In addition, DAR values calculated from SIL peptide mapping showed better linearity (R²=0.998) compared to standard peptide mapping and correlated within 5% of the theoretical DARs determined by HIC (FIG. 9).

Conjugation levels of belantamab mafodotin reference standard (DAR=4.0) were quantified and compared using both methods (Table 3). SIL peptide mapping preparations (n=4) were analyzed in parallel with standard peptide mapping preparations (n=4) to assess repeatability. Lower levels of light chain and heavy chain Fab conjugation (˜70%) were detected with SIL peptide mapping compared to standard peptide mapping (˜90%-95%). Higher levels of doubly-conjugated hinge (˜25%) and lower levels of singly-conjugated hinge (˜5%-10%) were detected compared to standard peptide mapping (˜15% and ˜12% for doubly and singly-conjugated, respectively). DAR values calculated from SIL peptide mapping (3.9-4.0) were also more accurate than those calculated from standard peptide mapping (4.6) when compared to the theoretical DAR of 4.0. Typical PTMs of belantamab mafodotin were also quantified, and no major differences were detected between the SIL and standard samples (Table 4). Complete sequence coverage was also detected for all samples.

TABLE 1
Belantamab Mafodotin (ADC) and Belantamab (mAb)
Combinations Used for Linearity Samples

| ADC (μg) | mAb (μg) | DAR |
|---|---|---|
| 0 | 250 | 0.0 |
| 12.5 | 237.5 | 0.3 |
| 25 | 225 | 0.6 |
| 50 | 200 | 1.1 |
| 100 | 150 | 2.3 |
| 150 | 100 | 3.4 |
| 200 | 50 | 4.6 |
| 250 | 0 | 5.7 |

TABLE 2
Conjugation Values and Calculated DARs for Belantamab
Mafodotin Linearity Samples

| | | | Conjugation (%) | HC Hinge | |
|---|---|---|---|---|---|
| | | | HC Hinge DL2 | DL1 | |
| | LC | HC Fab | (C230 and | (C230 or | Calc. |
| Sample | (C214) | (C224) | C233) | C233) | DAR |
| DAR 0.0 | 0.0 | 0.0 | 0.0 | 0.0 | 0.0 |
| DAR 0.3 | 5.0 | 5.7 | 2.9 | 1.0 | 0.4 |
| DAR 0.6 | 8.1 | 9.0 | 4.8 | 0.2 | 0.5 |
| DAR 1.1 | 18.5 | 20.3 | 11.7 | 1.1 | 1.3 |
| DAR 2.3 | 36.0 | 38.7 | 24.1 | 2.3 | 2.5 |
| DAR 3.4 | 55.0 | 57.0 | 36.9 | 4.7 | 3.8 |
| DAR 4.6 | 70.1 | 72.0 | 47.8 | 5.8 | 4.9 |
| DAR 5.7 | 84.5 | 85.9 | 57.9 | 7.7 | 5.9 |

TABLE 3
Comparison of Standard and SIL Peptide Mapping Conjugation
Values and Calculated DARs for Belantamab Mafodotin

| | | | Conjugation (%) | HC Hinge | |
|---|---|---|---|---|---|
| | LC | HC Fab | HC Hinge DL2 | DL1 (C230 | Calc. |
| Sample | (C214) | (C224) | (C230 and C233) | or C233) | DAR |
| Std. PM 1 | 94.7 | 92.9 | 15.5 | 12.1 | 4.6 |
| Std. PM 2 | 94.6 | 92.9 | 14.5 | 11.9 | 4.6 |
| Std. PM 3 | 94.8 | 92.8 | 14.8 | 12.3 | 4.6 |
| Std. PM 4 | 94.7 | 92.6 | 15.9 | 12.3 | 4.6 |
| SIL PM 1 | 67.9 | 71.5 | 26.4 | 5.7 | 4.0 |
| SIL PM 2 | 67.5 | 70.9 | 26.1 | 6.8 | 3.9 |
| SIL PM 3 | 67.7 | 71.1 | 26.0 | 9.5 | 4.0 |
| SIL PM 4 | 67.9 | 71.1 | 25.9 | 9.3 | 4.0 |

TABLE 4
Post-translational Modification Values for Belantamab
Mafodotin Samples using Standard and SIL Peptide Mapping

| | Deamidation | | Succinimide | | Isomer- | Suc- | | | | | | Pyro- | |
|---|---|---|---|---|---|---|---|---|---|---|---|---|---|
| | (%) | | (%) | | ization | cinimide | Oxidation (%) | | | | | glutamate | Truncation |
| | Asn | Asn | Asn | Asn | (%) | (%) | Met | Met | Met | Met | Met | (%) | (%) |
| Sample | 1 | 2 | 1 | 2 | Asp | Asp | 1 | 2 | 3 | 4 | 5 | Gln | Lys |
| Std | 1.9 | 0.8 | 0.9 | 0.2 | 4.3 | 0.8 | 0.1 | 0.2 | 2.4 | 0.4 | 0.2 | 100.0 | 90.7 |
| PM 1 | | | | | | | | | | | | | |
| Std. | 1.8 | 0.9 | 0.8 | 0.2 | 4.2 | 0.8 | 0.1 | 0.3 | 2.4 | 0.4 | 0.3 | 100.0 | 90.6 |
| PM 2 | | | | | | | | | | | | | |
| Std. | 2.1 | 0.8 | 0.8 | 0.2 | 4.5 | 0.8 | 0.1 | 0.2 | 2.4 | 0.4 | 0.3 | 100.0 | 91.2 |
| PM 3 | | | | | | | | | | | | | |
| Std. | 1.9 | 0.8 | 0.8 | 0.2 | 4.9 | 0.7 | 0.1 | 0.3 | 2.5 | 0.5 | 0.3 | 100.0 | 91.1 |
| PM 4 | | | | | | | | | | | | | |
| SIL PM | 1.2 | 0.4 | 0.5 | 0.1 | 3.9 | 0.7 | 0.1 | 0.3 | 3.0 | 0.3 | 0.4 | 100.0 | 90.9 |
| 1 | | | | | | | | | | | | | |
| SIL PM | 1.2 | 0.3 | 0.6 | <0.1 | 4.6 | 0.7 | 0.1 | 0.4 | 3.0 | 0.4 | 0.5 | 100.0 | 90.7 |
| 2 | | | | | | | | | | | | | |
| SIL PM | 1.3 | 0.5 | 0.7 | 0.1 | 4.6 | 0.8 | 0.2 | 0.4 | 2.9 | 0.4 | 0.4 | 100.0 | 90.7 |
| 3 | | | | | | | | | | | | | |
| SIL PM | 1.2 | 0.5 | 0.6 | <0.1 | 4.6 | 0.8 | 0.1 | 0.2 | 2.8 | 0.4 | 0.3 | 100.0 | 90.7 |
| 4 | | | | | | | | | | | | | |

### Example 4: Analysis of Hydrophobic Interaction Chromatography Drug-Load Fractions

### Sample Preparation

Differential DAR samples (DAR=2.1-5.7) and individual drug-load samples (DL2, DL4a, DL4b, and DL6) were analyzed with SIL peptide mapping as previously described in Experiment 3.

### Results and Discussion

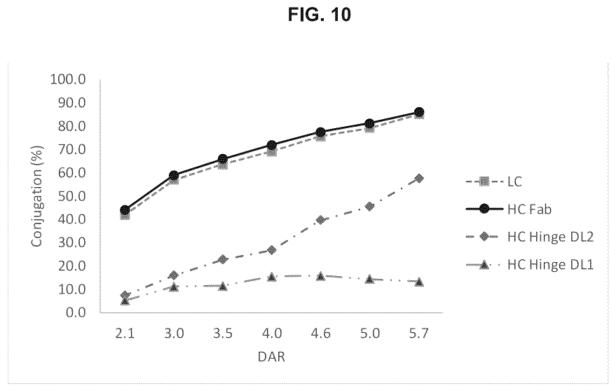

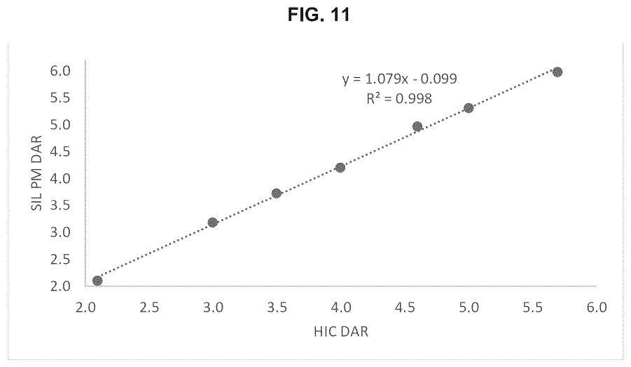

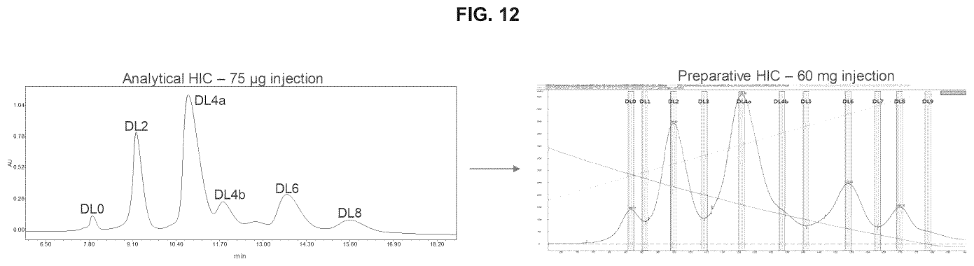

Belantamab mafodotin samples with DARs ranging from 2.1-5.7 were manufactured as part of a preclinical study to assess the impact of DAR on drug efficacy. DARs for these samples were calculated by integrating peak areas for the different drug load species in HIC chromatograms. SIL peptide mapping results (Table 5 and FIG. 10) showed comparable amounts of light chain and heavy chain Fab conjugation ranging from 41.8%-85.8% across increasing DAR samples. This result was expected due to the light chain and heavy chain Fab sites being disulfide-bond paired. Doubly-conjugated hinge showed a range from 7.2%-57.4% while singly-conjugated hinge resulted in a much smaller range from 5.1%
15.8%, highlighting the preference of the hinge to be doubly-conjugated at higher DARs. Interestingly, singly-conjugated hinge maximized in the 4.0 and 4.6 DAR samples showing the inflection point for reduced singly-conjugated hinge in higher DAR samples. DAR values calculated from HIC and SIL peptide mapping correlated within 10% showing the practicality of SIL peptide mapping for “bottom up” DAR characterization while also providing site-specific conjugation levels. (See FIG. 11)

Individual drug-load belantamab mafodotin samples (DL2, DL4a, DL4b, and DL6) were fraction-collected from a scaled-up hydrophobic interaction chromatography (HIC) method (FIG. 12). The major positional isomers for DL2, DL4, and DL6 were confirmed with orthogonal analytical methods and used to calculate theoretical conjugation site occupancy values. Theoretical values for the DL4b sample were not calculated due to this sample being a mixture of DL4a and DL4b isoforms due to HIC peak overlap. SIL peptide mapping produced values that correlated well with theoretical values (Table 6). SIL peptide mapping also confirmed the major form of hinge conjugation for isoforms DL4b and DL6 as doubly-conjugated while detecting minor amounts of singly-conjugated hinge.

TABLE 5
Conjugation Values and Calculated DARs for Belantamab
Mafodotin Differential DAR Samples

| | | | Conjugation (%) | HC Hinge | |
|---|---|---|---|---|---|
| | LC | HC Fab | HC Hinge DL2 | DL1 (C230 | Calc. |
| Sample | (C214) | (C224) | (C230 and C233) | or C233) | DAR |
| DAR 2.1 | 41.8 | 43.9 | 7.2 | 5.1 | 2.1 |
| DAR 3.0 | 56.8 | 58.9 | 15.8 | 11.1 | 3.2 |
| DAR 3.5 | 63.6 | 65.8 | 22.7 | 11.4 | 3.7 |
| DAR 4.0 | 69.1 | 71.8 | 26.7 | 15.3 | 4.2 |
| DAR 4.6 | 75.5 | 77.4 | 39.6 | 15.8 | 5.0 |
| DAR 5.0 | 79.1 | 81.0 | 45.4 | 14.3 | 5.3 |
| DAR 5.7 | 85.0 | 85.8 | 57.4 | 13.1 | 6.0 |

TABLE 6
Comparison of Theoretical and Experimental Conjugation
Values for Belantamab Mafodotin Drug-Load Samples
using SIL Peptide Mapping (theoretical based on structures)

| | Conjugation (%) | | | | | | | | |
|---|---|---|---|---|---|---|---|---|---|
| | LC | | HC Fab | | HC Hinge DL2 | | HC Hinge DL1 | | |
| | | SIL | | SIL | | SIL | | SIL | Calc. |
| Sample | Theoretical | PM | Theoretical | PM | Theoretical | PM | Theoretical | PM | DAR |
| DL2 | 50 | 51.0 | 50 | 51.8 | 0 | 0.0 | 0 | 0.3 | 2.1 |
| DL4a | 100 | 94.9 | 100 | 91.7 | 0 | 1.2 | 0 | 3.5 | 3.9 |
| DL4b | * | 49.9 | * | 53.1 | * | 48.3 | * | 6.3 | 4.1 |
| DL6 | 50 | 59.8 | 50 | 61.4 | 100 | 88.4 | 0 | 11.0 | 6.2 |

* Theoretical values not determined due to mixture

### Example 5: Non-Reduced Capillary Gel Electrophoresis (NR-CGE) Analysis of Drug-Load Variants

### Sample Preparation

A HIC preparative purification method was optimized and performed on an AKTA system using a Tosoh Toyopearl Butyl-650S column (35 μm, 8 mm×10 cm) (Part No. 45126) operating at 25° C. at a flow rate of 3.5 mL/min with a 60 mg injection and detection at 280 nm UV absorbance. The method uses a gradient flow of an initial mobile phase comprised of 1.5 M ammonium sulfate and 50 mM potassium phosphate at pH 7.0 and an elution mobile phase comprised of 20% 2-propanol and 50 mM potassium phosphate at pH 7.0. 4.5 mL fractions were collected at the peak apexes. The fractions for each DL variant (DL0, DL2, DL4a, DL4b, DL6, and DL8) were collected from multiple injections, pooled, and buffer exchanged into formulation buffer.

### Results

The purified DL variants, DL0, DL2, DL4a, DL4b, DL6, and DL8, were tested using the release and stability HIC method for purity analysis. Results obtained from HIC chromatograms of the DL variants are summarized in Table 7. Purity of DL0, DL2, DL4a, and DL6 was above 90%. DL4b and DL8 were 69.3% and 81.9% pure, respectively.

TABLE 7
HIC Results of Purified DL0, DL2, DL4a, DL4b, DL6, and DL8

| Sample | % DL0 | % DL1 | % DL2 | % DL3 | % DL4a | % DL4b | % DL5 | % DL6 | % DL8 | % DL10 |
|---|---|---|---|---|---|---|---|---|---|---|
| DL0 | 94.6 | 0.9 | 4.5 | ND | ND | ND | ND | ND | ND | ND |
| DL2 | ND | ND | 99.5 | 0.5 | ND | ND | ND | ND | ND | ND |
| DL4a | ND | ND | 1.0 | ND | 97.1 | 1.7 | ND | ND | 0.2 | ND |
| DL4b | ND | ND | 0.5 | 1.4 | 28.6 | 69.3 | ND | ND | 0.2 | ND |
| DL6 | ND | ND | 0.2 | ND | 1.0 | 3.2 | ND | 94.9 | 0.7 | ND |
| DL8 | ND | ND | 1.0 | ND | 0.8 | 3.9 | ND | 11.6 | 81.9 | 0.8 |

Note:
ND = Not Detected; Major DL variant in bold.

NR-CGE analysis of the purified DL variants was performed using the release and stability capillary gel electrophoresis (CGE) method. The denaturing sample preparation procedure leads to the dissociation of the heavy and light chains that are no longer connected by disulfide bonds, and the resulting separation provides a characteristic fingerprint of drug conjugation. The potential isoforms of belantamab mafodotin are shown schematically in FIG. 3 and the theoretical NR-CGE fingerprint of each DL variants is presented in Table 8.

TABLE 8
Theoretical Heavy and Light Chain Combinations for the
Potential Isoforms of Belantamab Mafodotin

| Drug Load Variant | Isoform | NR-CGE Fragments |
|---|---|---|
| DL0 | 0 | IgG |
| DL2 | 2a | HHLC, LC |
| | 2b | HHLL (IgG) |
| DL4 | 4a | HHC, LC |
| | 4b | HLC |
| | 4c | HHLC, LC |
| DL6 | 6a | HLC, HC, LC |
| | 6b | HHC, LC |
| DL8 | 8 | HC, LC |

Results for NR-CGE analysis of the purified DL variants are summarized in Table 9. Purified DL0 is 94.6% pure by HIC (Table 7) and 92.9% IgG by NR-CGE, indicating the DL0 peak in HIC is likely unconjugated IgG. Additional peaks in the NR-CGE profile of purified DL0 include light chain (LC) and HC—HC-LC (HHLC) species, which are likely from the 4.5% DL2 present in the fraction.

Purified DL2 contains predominantly LC and HHLC species in the NR-CGE analysis, which is consistent with DL2a and indicates the main DL2 variant is DL2a. Some DL2b co-elutes under the DL2 peak, suggested by the detection of 4.4% IgG in purified DL2.

Purified DL4a contains predominantly LC and HC—HC (HHC) species in the NR-CGE analysis indicating that DL4a is the predominant DL4 variant in belantamab mafodotin. The DL4a peak in HIC is mainly the DL variant conjugated at the four cysteine residues comprising the interchain disulphide bonds between the LC and HC. A HHLC fragment is observed at approximately 6% and is likely from either DL2a or DL4c. However, since the purity of DL4a is 97.1% by HIC, as shown in, there is potentially some co-elution of species under the DL4a peak which accounts for the higher HHLC content.

Purified DL4b contains predominantly LC, HHC, and HC-LC (HLC) species in the NR-CGE analysis. HIC results suggest 28.6% of the fraction is DL4a, which explains the observed LC and HHC fragments. The HLC fragment is a result of DL4b and indicates the species eluting under the DL4b peak in the HIC analysis of belantamab mafodotin is predominantly conjugated at the four cysteine residues comprising the two interchain disulfide bonds in the hinge region. Purified DL4b also contains a small amount of HHLC and HC fragments which is potentially from co-purification of DL4c or DL6a.

Purified DL6 contains a mixture of LC, HC, and HLC species in the NR-CGE analysis. This is consistent with DL6a and indicates the predominant DL6 variant in belantamab mafodotin is conjugated at the four cysteine residues comprising the two interchain disulfide bonds in the hinge region and the two cysteine residues from the LC and HC interchain disulfide bond. There is a HHC species present at 3.2%, potentially from co-purification of DL4a.

Purified DL8 contains predominantly LC and HC species, which is consistent with conjugation at the eight cysteine residues that comprise the four interchain disulfide bonds of belantamab mafodotin. A HLC fragment, potentially from co-purification of DL4b or DL6a, is present at approximately 11% and is consistent with the peaks reported in the HIC analysis (Table 7).

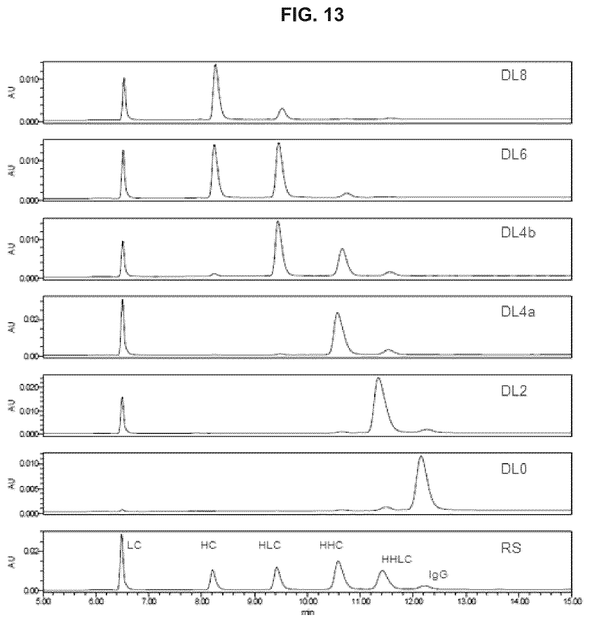

Representative NR-CGE electropherograms of purified DL0, DL2, DL4a, DL4b, DL6, and DL8 are demonstrated in FIG. 13.

TABLE 9
NR-CGE Results for Purified DL0, DL2, DL4a, DL4b, DL6, and DL8

| Sample | % LC | % HC | % HLC | % HHC | % HHLC | % IgG |
|---|---|---|---|---|---|---|
| DL0 | 1.7 | <LOQ | <LOQ | 1.4 | 4.0 | 92.9 |
| DL2 | 22.5 | <LOQ | <LOQ | 1.3 | 71.0 | 4.4 |
| DL4a | 39.2 | <LOQ | 0.9 | 53.6 | 6.1 | <LOQ |
| DL4b | 21.7 | 2.0 | 47.3 | 25.5 | 3.5 | <LOQ |
| DL6 | 23.0 | 35.3 | 38.4 | 3.2 | <LOQ | <LOQ |
| DL8 | 30.9 | 55.6 | 11.3 | 0.7 | 1.2 | <LOQ |

Note:
LOQ = Limit of Quantitation

### Example 6: Results from Intact and Reduced LC-MS

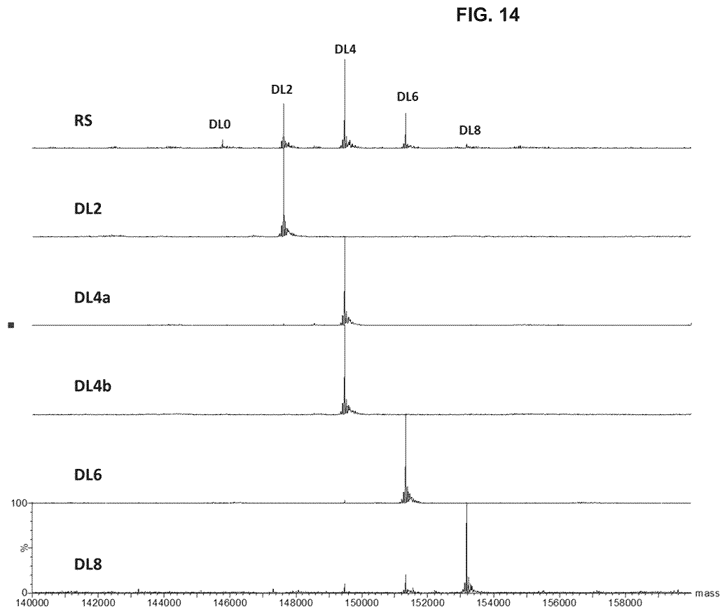

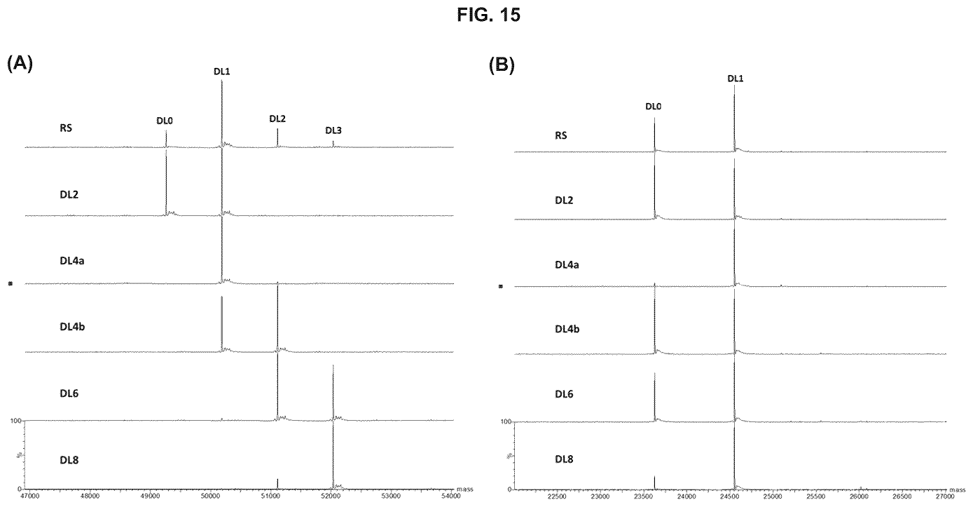

The purified DL variants prepared as described in Example 5, were analyzed using intact and reduced LC-MS. Total drug load in the purified DL variants was determined using intact LC-MS analysis. Total heavy chain and light chain drug loads were determined using reduced LC-MS. The results are shown in Table 10, and the spectra are shown in FIG. 14 and FIG. 15.

Results from intact LC-MS analysis of the purified DL variants correlate with the purity results from analytical HIC analysis (Table 7) in that similar species at approximately the same abundance are observed in both analyses. The exception to this is that a DL3 variant was detected by HIC in the purified DL4b fraction, but intact LC-MS analysis does not detect a species with a mass that correlates to a drug load of three drug molecules in this fraction. This suggests the DL3 peak in the purified DL4b fraction is not actually a species with a drug load of three drug molecules but another DL4 variant.

Reduced LC-MS analysis of the purified DL variants is consistent with the conjugation pattern predicted by NR-CGE results. Purified DL2 has a reduced LC-MS profile consistent with DL2a: 50% DL0 and 50% DL1 on both the LC and HC, indicating conjugation at the two cysteine residues comprising the interchain disulfide bond between the LC and HC.

Reduced LC-MS analysis of purified DL4a shows 93.6% and 96.9% DL1 on the LC and HC, respectively, and this is consistent with conjugation at the four cysteine residues comprising both interchain disulfide bonds between the LC and HC. This indicates purified DL4a is almost entirely DL4a variant. DL2a and DL4b are also observed at low levels in the HIC results and contribute to the low levels of observed LC DL0 and HC DL2 in the reduced LC-MS analysis.

Reduced LC-MS of a pure DL4b variant is expected to consist of 100% LC DL0 and 100% HC DL2. LC DL0 is present at 50.8%, and HC DL2 is present at 54.7% indicating that approximately 50% of the sample contains DL4b. Because 50% of the LC is DL1 and 50% of the HC is DL1 indicate there is likely DL4a in the population, which is consistent with the 30% DL4a detected by HIC. The other DL4 variants may contribute to the LC DL1 and HC DL1 peaks detected in the reduced LC-MS analysis.

Reduced LC-MS analysis of purified DL6 indicate the purified DL6 fraction is variant DL6a. A pure DL6a variant is expected to consist of 50:50 DL0:DL1 on the LC and 50:50 DL2: DL3 on the HC. There is slightly more LC DL1 and HC DL2 than expected from a pure DL6a fraction, which potentially indicates there is some DL6b variant present. These results indicate the predominant DL6 variant in belantamab mafodotin is conjugated at the four cysteine residues that comprise the interchain disulfide bonds in the hinge and at the two cysteine residues comprising the interchain disulfide bond between the LC and HC.

Reduced LC-MS analysis of purified DL8 is expected to consist of 100% LC DL1 and 100% HC DL3. The results indicate that greater than 80% of LC is DL1 and greater than 80% of HC is DL3, and these results are consistent with the expected abundance of approximately 80% based on HIC purity (Table 7). LC DL1 and HC DL2 are detected at 17% and 14%, respectively, and are likely the result of the DL6 and DL4b co-purifying with the DL8 variant based on the HIC results.

TABLE 10
Intact and Reduced LC-MS Results of Purified DL0, DL2, DL4a, DL4b, DL6, and DL8

| | | | RS Lot 182407660 | | | DL2 | | | DL4a | | |
|---|---|---|---|---|---|---|---|---|---|---|---|
| | | | | ME | Abundance | | ME | Abundance | OM | ME | Abundance |
| Analyte | Variant | TM (Da) | OM (Da) | (Da) | (%) | OM (Da) | (Da) | (%) | (Da) | (Da) | (%) |
| Intact | DL0 | 145772 | 145772 | 0 | 4.8 | Not Detected (ND) | | | ND | | |
| | DL2 | 147623 | 147627 | 4 | 24.4 | 147629 | 6 | 100.0 | | | |
| | DL4 | 149473 | 149481 | 8 | 49.1 | ND | | | 149481 | 8 | 100.0 |
| | DL6 | 151323 | 151333 | 10 | 19.3 | | | | ND | | |
| | DL8 | 153174 | 153185 | 11 | 2.5 | | | | | | |
| Light | DL0 | 23628 | 23628 | 0 | 34.0 | 23628 | 0 | 52.3 | 23628 | 0 | 6.4 |
| Chain | DL1 | 24554 | 24553 | −1 | 66.0 | 24553 | −1 | 47.7 | 24553 | −1 | 93.6 |
| Heavy | DL0 | 49262 | 49261 | −1 | 15.7 | 49262 | 0 | 49.3 | ND | | |
| Chain | DL1 | 50187 | 50187 | 0 | 60.4 | 50187 | 0 | 50.7 | 50187 | 0 | 96.9 |
| | DL2 | 51112 | 51112 | 0 | 17.2 | ND | | | 51112 | 0 | 3.1 |
| | DL3 | 52037 | 52038 | 1 | 6.7 | | | | ND | | |
| | | | DL4b | | | DL6 | | | DL8 | | |
| | | | OM | ME | Abundance | OM | ME | Abundance | OM | ME | Abundance |
| Analyte | Variant | TM (Da) | (Da) | (Da) | (%) | (Da) | (Da) | (%) | (Da) | (Da) | (%) |
| Intact | DL0 | 145772 | ND | | | ND | | | ND | | |
| | DL2 | 147623 | | | | | | | | | |
| | DL4 | 149473 | 149480 | 7 | 100.0 | | | | 149479 | 6 | 8.0 |
| | DL6 | 151323 | ND | | | 151333 | 10 | 100.0 | 151332 | 9 | 15.7 |
| | DL8 | 153174 | | | | ND | | | 153184 | 10 | 76.3 |
| Light | DL0 | 23628 | 23628 | 0 | 50.8 | 23628 | 0 | 42.1 | 23628 | 0 | 17.3 |
| Chain | DL1 | 24554 | 24553 | −1 | 49.2 | 24553 | −1 | 57.9 | 24552 | −2 | 82.7 |
| Heavy | DL0 | 49262 | ND | | | ND | | | | | |
| Chain | DL1 | 50187 | 50187 | 0 | 45.3 | 50184 | −3 | 2.1 | ND | | |
| | DL2 | 51112 | 51112 | 0 | 54.7 | 51112 | 0 | 53.8 | 51111 | −1 | 13.7 |

| | | | | | | | | |
|---|---|---|---|---|---|---|---|---|
| DL3 | 52037 | ND | 52037 | 0 | 44.1 | 52036 | −1 | 86.3 |

Note:
ME = Mass Error; ND = Not detected; OM = Observed Mass; TM = Theoretical Mass

### Example 7: Peptide Mapping by LC-MS/MS

The conjugation sites and post-translational modifications of the purified DL variants prepared as described in Example 5, were evaluated using peptide mapping LO-MS/MS.

### Antigen, FcγRIIIa, and FcRN Binding by SPR

Antigen, FcγRIIIa, and FcRn specific binding activity of the purified DL variants was measured using SPR. Antigen, FcγRIIIa and FcRN analyses were performed using a release and stability surface plasmon resonance (SPR) method. The specific binding activities measured were between 80-110% for the purified DL variants. The three SPR activity assays showed a decrease in specific binding as the number of conjugated drug molecules increased (Table 11). Despite the trend observed, the decrease in binding activity as a function of drug load is minimal and remains within specification acceptance criteria. It has been observed that as the DAR increases, there is no significant change in antigen FcγRIIIa binding by SPR and antibody-dependent cell-mediated cytotoxicity (ADCC) activity.

TABLE 11
Specific Binding Activity of Purified DL0, DL2,
DL4a, DL4b, DL6, and DL8

| | Antigen | FcyRIIIa | FcRNn |
|---|---|---|---|
| Sample | Binding (%) | Binding (%) | Binding (%) |
| Control | 96 | 97 | 101 |
| DL0 | 103 | 110 | 108 |
| DL2 | 99 | 104 | 109 |
| DL4a | 95 | 103 | 102 |
| DL4b | 90 | 91 | 99 |
| DL6 | 89 | 84 | 97 |
| DL8 | 86 | 82 | 93 |

### Potency by Cell Growth Inhibition and ADCC Reporter Bioassays

Biological activity of the purified DL variants was monitored using a release and stability cell growth inhibition bioassay and the ADCC reporter bioassay. The results are shown in Table 12. The relative potency of the purified DL variants in the cell growth inhibition bioassay correlated with an increase in drug load. The relative potency of the purified DL0 variant in the cell growth inhibition bioassay was 0.0 as expected for an unconjugated molecule.

The relative potency of the purified DL variants in the ADCC reporter bioassay was similar. There is potentially a correlation between increased relative potency and a decrease in drug load, similar to what was observed in the FcγRIIIa and FcRn binding (Table 11). This is not unexpected because conjugation of drugs near the hinge region could cause minor conformational changes that impact protein-protein interactions in the Fc.

TABLE 12
Relative Potency of Purified DL0, DL2, DL4a, DL4b, DL6, and DL8
using Cell Growth Inhibition and ADCC Reporter Bioassays

| | Cell Growth Inhibition | ADCC Reporter Bioassay |
|---|---|---|
| Sample | (Relative Potency) | (Relative Potency) |
| DL0 | 0.0 | 1.1 |
| DL2 | 0.5 | 1.3 |
| DL4a | 0.9 | 1.3 |
| DL4b | 1.0 | 1.0 |
| DL6 | 1.6 | 0.9 |
| DL8 | 1.8 | 0.7 |

### Capillary Differential Scanning Calorimetry (DSC)

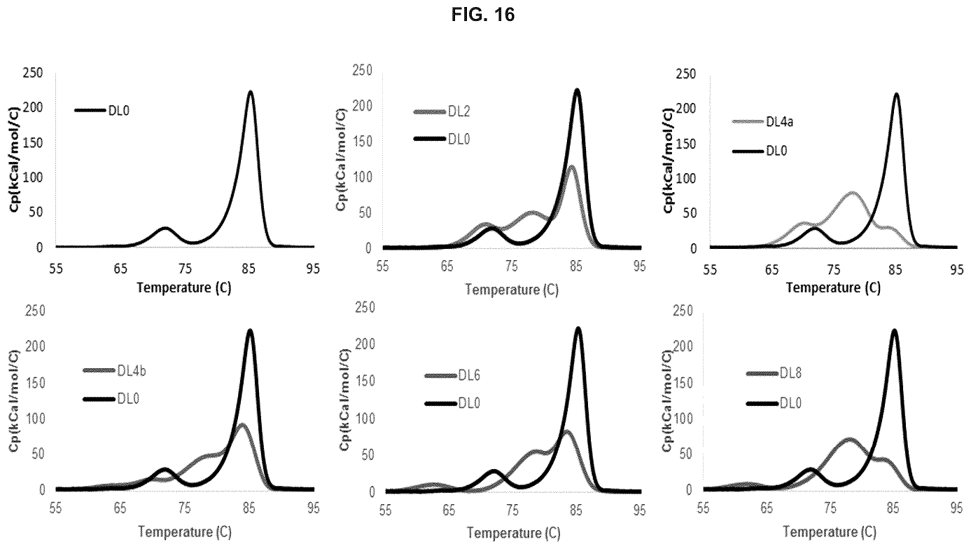

A capillary DSC method was used to measure changes in belantamab mafodotin tertiary structure and thermal stability after drug conjugation (FIG. 16). In a typical thermogram of belantamab, the first peak corresponds to the unfolding of the CH2 domain, and the second peak corresponds to the unfolding of the CH3 domain and Fab. The CH3 and Fab melting transitions of mAbs often overlap and typically occur at similar or higher temperatures when compared to that of the CH2 domain. Melting temperatures generally correlate with protein stability; increasing stability is usually reflected by higher melting temperatures as the amount of energy required to unfold the protein increases.

Table 13 and FIG. 16 present the transition temperatures and DSC thermograms, respectively, of the purified DL variants. The DSC thermogram and transition temperatures of purified DL0 are similar to that of belantamab. A small increase in Tm₂ is observed when compared to belantamab, which is possibly due to the differences in formulation buffers.

DSC analysis of purified DL2 shows a decrease in apparent transition temperature of the Fab from 84.2° C. to 78.2° C. This is likely the result of drug conjugation on LC C214 and HC C224, which are the cysteine residues that form the interchain disulphide bond between the LC and HC. This data is consistent with the NR-CGE and reduced LC-MS results.

DSC analysis of purified DL4a is similar to what is observed in purified DL2 and shows a decrease in the apparent transition temperature of the Fab. However, when compared to purified DL2, the purified DL4a variant has a much larger peak area at 78.0° C. This correlates with the data that show that both cysteines that comprise the interchain disulphide bond between the LC and HC are conjugated. A slight decrease in the apparent transition temperature of the CH2 domain is also observed which is potentially from another DL4 variant that co-purified with DL4a.

DSC analysis of purified DL4b appears similar in part to the results of purified DL2: there is a decrease in the apparent transition temperature of the Fab domain. Unlike DL2, however, there is also a decrease in the apparent transition temperature of the CH2 domain. This suggests purified DL4b contains species with conjugation at cysteines in both the hinge and Fab interchain disulfides.

DSC analysis of purified DL6 shows a decrease in the apparent transition temperature of the CH2 domain and a portion of the Fab. The proportion of the Fab that has shifted is similar to what is observed in purified DL2. This data is consistent with drug conjugation of the two cysteine residues that form the interchain disulfide bond between the LC and HC, and the four cysteine residues in the hinge interchain disulfide bonds.

DSC analysis of purified DL8 shows a decrease in the melting temperature of the CH2 domain similar to what is observed for purified DL6. Additionally, there is a decrease in the apparent transition temperature the Fab that is similar to what was observed in purified DL4a. These results are consistent with conjugation of the eight cysteine residues in the four interchain disulfide bonds.

Disruption of the interchain disulfide bonds through partial reduction and conjugation of the cysteine residues is expected to lead to lower transition temperatures of the thermal unfolding. The capillary DSC data generated on the purified DL variants are consistent with this hypothesis.

TABLE 13
Capillary DSC Results of HIC Purified DL0, DL2, DL4a,
DL4b, DL6, and DL8

| | Total Enthalpy | Tm₁ | Tm₂ | Tm₃ | Tm₄ |
|---|---|---|---|---|---|
| Sample | (kCal/mole) | (° C.) | (° C.) | (° C.) | (° C.) |
| DL0 | 1102.6 | ND | 71.9 | ND | 85.2 |
| DL2 | 972.9 | ND | 70.9 | 78.2 | 84.2 |
| DL4a | 918.3 | ND | 70.9 | 78.0 | 83.7 |
| DL4b | 874.4 | ND | 70.0 | 78.8 | 83.9 |
| DL6 | 849.4 | 62.5 | ND | 78.5 | 83.3 |
| DL8 | 812.2 | 61.8 | ND | 78.0 | 84.1 |

Notes:
ND = Not detected

Conclusions from Examples 5-7

Purified DL variants were characterized to determine their purity, potency, and identity, including the sites of conjugation on belantamab mafodotin. The results show that the DL distribution is a heterogenous mixture of DL variants conjugated with even numbers of drug (mcMMAF) after partial reduction of the interchain disulfide bonds. The main drug load variant of belantamab mafodotin is the DL4a species and contains four MMAF molecules conjugated at each LC C214 and each HC C224 comprising the interchain disulfide bonds between the LC and HC. The DL0 variant was confirmed to be unconjugated belantamab. While the relative potency of the DL variants increased in the cell growth inhibition assay in relation to increasing drug load, belantamab mafodotin is comprised of a distribution of these DL variants that contribute to the overall relative potency. The DL distribution will be controlled by the drug-antibody ratio (DAR). The HIC method is currently in place for both drug substance and drug product to monitor the drug load variants of belantamab mafodotin at release and on stability.

1. An analytical method comprising:
(i) conjugation of unoccupied cysteine sites of a cysteine-conjugated antibody drug conjugate (ADC) using an isotopically-labeled cytotoxin containing a carbonyl group and a reductant to produce an isotopically-labeled ADC sample; and
(ii) peptide mapping the sample.

2. The method of claim 1, wherein the cytotoxin is MMAF or MMAE.

3. The method of claim 1, wherein the ADC is first reduced by the reductant and then conjugated with the isotopically-labeled cytotoxin.

4.-6. (canceled)

7. The method of claim 1, wherein excess isotopically-labeled cytotoxin is removed prior to the peptide mapping by eluting the sample through a size exclusion chromatography column.

8. The method of claim 1, wherein the peptide mapping comprises using liquid chromatography tandem mass spectrometry (LC-MS/MS) analysis.

9. The method of claim 1, wherein the peptide mapping comprises denaturing the sample, reducing all remaining disulfide bonds, and alkylating resulting free sulfhydryls.

10. The method of claim 1, wherein the peptide mapping comprises enzymatically digesting the sample to produce isotopically-labeled conjugated peptides and optionally quenching the enzymatic digestion by addition of a strong acid.

11.-14. (canceled)

15. The method of claim 1, wherein the ADC is belantamab mafodotin.

16.-19. (canceled)

20. A composition comprising an anti-BCMA antibody conjugated to a cytotoxic agent to form an antibody drug conjugate (ADC), wherein the antibody comprises a CDRH1 comprising the amino acid sequence according to SEQ ID NO:1; a CDRH2 comprising the amino acid sequence according to SEQ ID NO:2; a CDRH3 comprising the amino acid sequence according to SEQ ID NO:3; a CDRL1 comprising the amino acid sequence according to SEQ ID NO:4; a CDRL2 comprising the amino acid sequence according to SEQ ID NO:5; and a CDRL3 comprising the amino acid sequence according to SEQ ID NO:6; wherein the cytotoxic agent is MMAF or MMAE; and wherein the percentage drug load at LC C214 is between about 56% to about 80%, the percentage drug load at HC C224 is between about 58% to about 81%, the percentage drug load of HC hinge DL2 at HC C230 and C233 is between about 15% to about 46%, and/or the percentage drug load of HC hinge DL1 at HC C230 or HC C233 is between about 11% to about 15%.

21.-26. (canceled)

27. A pharmaceutical composition comprising the composition of claim 20 and at least one pharmaceutically acceptable excipient.

28. A method of treating cancer comprising administering to a subject in need thereof a therapeutically effective amount of the composition of claim 20.

29.-30. (canceled)

31. A method of determining conjugation levels of cysteine-conjugated antibody drug conjugates, the method comprising:
a) reducing the antibody drug conjugates to form reduced antibody drug conjugates;
b) conjugating the reduced antibody drug conjugates with an isotopically-labeled cytotoxin to form isotopically-labeled antibody drug conjugates;
c) producing isotopically-labeled conjugated peptides from the isotopically-labeled antibody drug conjugates and performing peptide mapping on the isotopically-labeled conjugated peptides;
d) detecting mass-to-charge ratios for the isotopically-labeled conjugated peptides; and
e) comparing the mass-to-charge ratios of the isotopically-labeled conjugated peptides to mass-to charge ratios for non-isotopically-labeled conjugated peptides to determine the conjugation levels of cysteine-conjugated antibody drug conjugates.

32. The method of claim 31, wherein the cytotoxin is MMAF or MMAE.

33. The method of claim 31, wherein the cysteine-conjugated antibody drug conjugates are first reduced by a reductant and then conjugated with the isotopically-labeled cytotoxin.

34. (canceled)

35. The method of claim 33, wherein excess reductant is removed prior to the peptide mapping by eluting the sample through a size exclusion chromatography column.

36.-37. (canceled)

38. The method of claim 31, wherein the peptide mapping comprises using liquid chromatography tandem mass spectrometry (LC-MS/MS) analysis.

39. The method of claim 31, wherein the peptide mapping comprises denaturing the sample, reducing remaining disulfide bonds, and alkylating resulting free sulfhydryls.

40. The method of claim 31, wherein the peptide mapping comprises enzymatically digesting the sample to produce isotopically-labeled conjugated peptides and optionally quenching the enzymatic digestion by addition of a strong acid.

41. The method of claim 31, wherein the method comprises reacting cytotoxin with isotopically-labeled water to produce the isotopically-labeled cytotoxin.

42. (canceled)

43. The method of claim 31, wherein the cysteine-conjugated antibody drug conjugates are belantamab mafodotin.
